(19) United States
(12) Patent Application Publication
(10) Pub. No.: US 2020/0170950 A1
(43) Pub. Date: Jun. 4, 2020

(54) COMPOSITIONS COMPRISING A CANNABINOID OR A CANNABIS-DERIVED COMPOUND, METHODS OF MAKING AND USE

(71) Applicant: CANOPY GROWTH CORPORATION, Smith Falls (CA)

(72) Inventors: Chris ADAIR, Smith Falls (CA); Ben GEILING, Smith Falls (CA); Mohammadmehdi Haghdoost MANJILI, Smith Falls (CA); Mark ZOLKIEWSKI, Smith Falls (CA)

(21) Appl. No.: 16/699,276

(22) Filed: Nov. 29, 2019

Related U.S. Application Data
(60) Provisional application No. 62/773,616, filed on Nov. 30, 2018.
(60) Provisional application No. 62/773,639, filed on Nov. 30, 2018.

(51) Int. Cl. A61K 9/14; A61K 31/05; A61K 31/352; A61K 47/44; A61K 9/107; A61K 9/00; A61K 47/36; A23L 33/105; A23L 2/52

(52) U.S. Cl. CPC A61K 9/146; A61K 31/05; A61K 31/352; A61K 47/44; A23V 2002/00; A61K 9/0095; A61K 47/36; A23L 33/105; A23L 2/52; A61K 9/107

(57) ABSTRACT

Cannabis and cannabis-derived compositions, methods of making and methods of using the compositions in oral dosage forms, such as in beverage forms, are disclosed herein. The compositions comprise a cannabinoid or a cannabis-derived compound, inulin and pectin and may be in powder or liquid form.

### CROSS-REFERENCE TO RELATED APPLICATIONS

This application claims the benefit of and priority to U.S. Patent Application Nos. 62/773,616 and 62/773,639 filed on Nov. 30, 2018, each of which are hereby incorporated by reference in their entirety.

### TECHNICAL FIELD

The present disclosure generally relates to compositions comprising cannabinoids or cannabis-derived compounds that may be used in the preparation of beverages, foodstuffs and other products. In particular, the present disclosure relates to compositions comprising a cannabinoid or a cannabis-derived compound, inulin and pectin.

### BACKGROUND

In the cannabis industry, an important aspect of preparing a commercial product is the ability to formulate cannabinoids and other cannabis-derived compounds in a desirable form for human consumption.

Smoking is not typically acceptable to non-smokers, as it can be aesthetically unpleasant and can involve health risks such as irritation to at least the mouth, esophagus and lungs. Cigarette smoking has been linked to devastating health risks thought to result from the formation of harmful combustion products. In some jurisdictions, legislation exists which prohibits smoking in various locations and cannabis smoking itself is the target of regulation due to so-called “second hand smoke” risks, as well as what is said to be unpleasant smells for some people. Methods for consuming cannabis, and more particularly cannabinoids, which do not involve smoking or other vaporous means of ingestion may therefore be advantageous as such methods do not involve these and other unwanted effects.

Oral consumption comprises a significant percentage of total cannabis use in federally legal jurisdictions as well as on a state, province, or the like, basis globally. Many orally consumable products, however, contain unhealthy amounts of substances other than cannabis or cannabinoids. Such ingredients include various sugars, caffeine and a variety of non-sugar stimulants, ethanol, and plant-based substances thought to be nutritional supplements, but which have not been the subject of extensive safety testing in complex formulations including cannabis and cannabinoid-containing compositions. Further, many known oral products use expensive gums, which are cost prohibitive and may also have unpredictable supply.

As hydrophobic compounds, cannabinoids and other cannabis-derived compounds present challenges for preparing desirable consumer products, such as beverages and other foodstuffs. Cannabinoids, including many cannabinoid extracts and oils, are insoluble in water thereby making many food products and beverages difficult to produce, including difficulties in obtaining desirable concentrations of cannabinoids in these products.

A need therefore exists for improved compositions of cannabinoids that may be used in the preparation of consumer products, and in particular aqueous-based products such as beverages. There further exists a need that these compositions be easy to prepare at relatively inexpensive costs, and have wide-range applicability in preparing consumer products.

### SUMMARY

The present disclosure provides a convenient water-soluble composition of cannabinoids or cannabis-derived compounds that may be used in beverages and foodstuffs. More particularly, in select embodiments, the present disclosure provides a composition of cannabinoids in liquid, powder and solid forms that is soluble in water, of natural origin and calorie-free (e.g., less than 5 kcal per serving), and that has little or no taste and odor. In some aspects, as an alternative or in addition to the cannabinoids, the compositions of the present disclosure may include other cannabis-derived compounds (e.g., cannabis extract, terpenes, etc.), non-cannabis-derived compounds (e.g. non-cannabis terpenes), and/or nutritional supplements (e.g., vitamins) in a single convenient composition or dosage form.

The present disclosure is directed to compositions comprising a cannabinoid or a cannabis-derived compound, methods of making such compositions, and uses thereof. The present disclosure is also directed to foodstuffs and beverages comprising said compositions (e.g. produced using such compositions). In particular, the compositions of the present disclosure comprise a cannabinoid or a cannabis-derived compound, inulin and pectin. The compositions may be powder or liquid formulations.

Most suitably and in select embodiments, the compositions are physically and chemically stable; transparent, translucent or pearlescent in colour; calorie-free; comprised of natural ingredients; and have minimal flavor. Further, in select embodiments, the compositions include favorable pharmacokinetics, for example, rapid onset, shorter duration, and minimal food effect as described more fully herein. The present disclosure is also directed to methods of preparing the compositions that are commercially-viable, efficient, and produce stable compositions with a high loading of cannabinoids.

According to a first aspect of the present disclosure, there is provided a composition comprising a cannabinoid or a cannabis-derived compound, inulin and pectin. In select embodiments, the pectin is a sugar beet pectin. In alternative embodiments, the pectin is a citrus pectin. In other embodiments, the pectin is a combination of sugar beet pectin and citrus pectin.

In some embodiments, the inulin and pectin are present in the composition in a ratio of inulin:pectin of between about 99%:1% w/w to about 60%:40% w/w, sometimes more particularly in a ratio of inulin:pectin of between about 95%:5% to about 70%:30% w/w.

In some embodiments, the inulin and pectin are present in the composition in a ratio of inulin:pectin of about 95%:5% w/w. In alternative embodiments, the inulin and pectin are present in the composition in a ratio of inulin:pectin of about 70%:30% w/w.

In some embodiments, the cannabinoid is THC (Δ9-THC), Δ8-THC, trans-Δ10-THC, O-THC, THCA, THCV, Δ8-THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, CBT or any combination thereof. In select embodiments, the cannabinoid is CBD, THC or a combination thereof. In select embodiments, the cannabinoid is THC alone or CBD alone.

In some embodiments, the composition may further comprise a terpene, a terpenoid, a flavonoid, a viscosity modifier, a nutritional supplement, or any combination thereof.

In some embodiments, the compositions of the present disclosure are in liquid form. For example, the composition may be a liquid formulation and further comprise a solvent. In select embodiments, the solvent is water and the liquid formulation is an emulsion. The emulsion may be an oil-in-water emulsion.

In other embodiments, the compositions of the present disclosure are in solid form, such as in powder form or granule form. In an embodiment, the powder form is configured to be added directly to foodstuffs and liquid beverages. Thus, in an embodiment, the composition herein may be a powder formulation. The powder formulation may further comprise a bulking agent. In select embodiments, the bulking agent may comprise a sugar alcohol, such as myo-inositol.

In some embodiments, the compositions of the present disclosure comprise core-shell structures comprising a hydrophobic inner core comprising the cannabinoid or cannabis-derived compound and a hydrophilic outer shell comprising the inulin and pectin. In select embodiments, the ratio of the hydrophilic outer shell:hydrophobic inner core may be between about 90%:10% w/w to about 50%:50% w/w. In some embodiments, the ratio of hydrophilic outer shell:hydrophobic inner core is about 80%:20% w/w. This ratio may be preferred for compositions comprising THC as a cannabinoid. In some embodiments, the ratio of hydrophilic outer shell:hydrophobic inner core is about 75%:25% w/w. This ratio may be preferred for compositions comprising CBD as a cannabinoid.

In some embodiments, the hydrophobic inner core may further comprise a carrier solvent. The carrier solvent may be any suitable solvent capable of mixing with, or dissolving, the cannabinoid or cannabis-derived compound. In an embodiment, the carrier solvent is an oily medium. In select embodiments, the carrier solvent may comprise coconut oil or medium-chain triglyceride (MCT) oil.

In some embodiments, the composition of the present disclosure may comprise a weight ratio of cannabinoid:carrier solvent of between about 3:1 to about 1:3 (i.e. 75%:25% w/w to 25%:75% w/w). In some embodiments, the ratio of cannabinoid:carrier solvent is about 1:1.

In some embodiments, the composition of the present disclosure comprises core-shell structures comprising a hydrophobic inner core comprising the cannabinoid and a hydrophilic outer shell comprising the inulin and pectin, wherein: the inulin and pectin are present in a ratio of about 70%:30% w/w inulin:pectin; the core-shell structures comprise a ratio of about 80%:20% w/w hydrophilic outer shell:hydrophobic inner core; and the hydrophobic inner core further comprises a carrier solvent at a ratio of about 1:1 w/w cannabinoid:carrier solvent. In select embodiments, such composition is a powder formulation.

According to a second aspect of the present disclosure, there is provided a method of preparing the compositions disclosed herein, the method comprising combining the cannabinoid or the cannabis-derived compound with inulin and pectin; and homogenizing the mixture with a solvent to form an emulsion.

In a particular embodiment, the method of preparing the compositions disclosed herein comprises: combining the inulin and pectin in the solvent to form an inulin/pectin mixture; combining the cannabinoid or the cannabis-derived compound with the inulin/pectin mixture; and homogenizing the mixture to form an emulsion. In select embodiments, the solvent is water. The inulin and pectin may be dissolved in the solvent at any suitable quantity. In an embodiment, the inulin and pectin are dissolved in the solvent at a concentration of between about 4% w/w to about 15% w/w. In an embodiment, the inulin and pectin are combined in a ratio of between about 95%:5% to about 70%:30% w/w.

In some embodiments, the method further comprises mixing the cannabinoid or the cannabis-derived compound in a carrier solvent prior to combining the cannabinoid or the cannabis-derived compound with the inulin/pectin mixture. The carrier solvent may be any solvent suitable for mixing with, or dissolving, the cannabinoid or cannabis-derived compound. In select embodiments, the carrier solvent may comprise coconut oil or MCT oil.

In some embodiments, the step of homogenizing may comprise one or more of: magnetic stirring, high-shear mixing, microfluidizing, sonication, and ultrasonication.

In some embodiments, the method may further comprise drying the emulsion to form a powder. The step of drying may comprise any process suitable for removing solvent from the liquid emulsion to form a powder formulation, for example, spray drying, freeze drying, drum drying, pulse combustion drying, pan coating, air-suspension coating, centrifugal extrusion, vibrational nozzle technique, and/or use of a food dehydrator. In some embodiments, the drying comprises spray drying.

In some embodiments, the methods disclosed herein provide a powder that comprises core-shell structures comprising a hydrophobic inner core comprising the cannabinoid or cannabis-derived compound and a hydrophilic outer shell comprising the inulin and pectin. In some embodiments, at least 70% by weight of total cannabinoid or cannabis-derived compound that was combined with the inulin and pectin is encapsulated in the hydrophobic inner core (e.g. at least 70% encapsulation efficiency). In some embodiments, at least 95% by weight of total cannabinoid or cannabis-derived compound that was combined with the inulin and pectin is encapsulated in the hydrophobic inner core (e.g. at least 95% encapsulation efficiency).

In a third aspect of the present disclosure, there is provided a composition prepared according to the methods disclosed herein.

In a fourth aspect of the present disclosure, there is provided a foodstuff comprising or prepared with the composition disclosed herein, or the composition prepared according to the methods disclosed herein.

In a fifth aspect of the present disclosure, there is provided a beverage comprising or prepared with the composition disclosed herein, or the composition prepared according to the methods disclosed herein.

In a sixth aspect of the present disclosure, there is provided a dosage form comprising or prepared with the composition disclosed herein, or the composition prepared according to the methods disclosed herein.

Other aspects and features of the compositions, methods and products (e.g. dosage forms, beverages and foodstuffs) of the present disclosure will become apparent to those ordinarily skilled in the art upon review of the following description of specific embodiments. Without being bound by any particular theory, the compositions of the present disclosure may improve the ability to formulate cannabinoids into aqueous mediums (e.g. beverages and foodstuffs).

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 depicts an exemplary powder formulation of the present disclosure.

FIGS. 2A & 2B depict the powder formulation of FIG. 1 viewed directly under a microscope (Zeiss Stemi DV4), 8× magnification.

FIGS. 2C & 2D depict the powder formulation of FIG. 1 viewed directly under a microscope (Zeiss Stemi DV4), 32× magnification.

FIG. 3 depicts an exemplary liquid formulation of the present disclosure.

FIGS. 4A & 4B depict the powder formulation of FIG. 1 dissolved in water and placed on a microscope slide (Zeiss, Axio Lab.A1, 10× ocular lens and 100× objective lens=1000× magnification).

FIG. 5 depicts a simplified flow chart exemplifying process steps for preparing compositions of the present disclosure.

FIGS. 6A & 6B depict THC concentration in brewed tea for a tea bag dosed with a powder formulation according to the disclosure (FIG. 6B), compared to a control of the same powder formulation in boiling water (FIG. 6A).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the suitable methods and materials are described below.

The present disclosure is generally directed to compositions comprising a cannabinoid or a cannabis-derived compound, inulin and pectin, methods for their preparation and use thereof. The compositions are suitably nontoxic consumable liquid or powder forms, such as the powder formulations and liquid formulations disclosed herein. Suitably, embodiments of the compositions disclosed herein provide stability, solubility in water, have minimal flavor and odor, are calorie-free, and are natural in origin. In some embodiments, the compositions can contain flavor, odor and/or calories if desired, particularly when comprised in or used for the preparation of a beverage or foodstuff.

As mentioned above, the compositions of the present disclosure may be a solid or liquid form. For example, the compositions may be provided as a powder formulation or as a liquid formulation. As used herein, the term “composition” is meant to refer broadly to any product or material comprised of two or more components (e.g. a cannabinoid or cannabis -derived compound, inulin and pectin). A “formulation” is more narrowly meant to define the composition according to a particular physical state (e.g. powder or liquid) or the act, process or result of formulating according to a particular formula. In the context of the present disclosure, any features described for formulations of the present disclosure also apply to compositions, and vice versa.

The compositions of the present disclosure include a cannabinoid or a cannabis-derived compound. Cannabis has been used in beverage preparations for years. Most of the historical cannabis beverages were prepared by boiling or grinding cannabis leaves, combining with water, milk, alcohol, or another biocompatible matrix or beverage liquid and, optionally, mixing with herbal or other plant-based compositions to form the final consumable.

The present disclosure provides improved compositions for cannabinoids and cannabis-derived compounds (e.g. cannabis concentrate, terpenes, etc.). As shown herein, the compositions of the present disclosure comprising cannabinoids, inulin and pectin are highly soluble in water (e.g. Example 3). Thus, the present disclosure provides convenient water-soluble compositions of cannabinoids that may be readily used in the preparation of beverages and foodstuffs.

The liquid formulations of the present disclosure show high emulsion stability, evidenced both by the observed stability over time under various parameters, such as different inulin:pectin ratios, core:wall ratios, and/or cannabinoid concentrations, as well as an observed low quantity of oil droplets and oil droplets being of small size (Example 5). Spray-dried powder formulations likewise show advantageous properties, such as high encapsulation efficiency of cannabinoids, normal distributions on cyclone spray drying (often with minimal or no build up within the dryer), and good colour/consistency as white or slightly beige powders (e.g. Examples 6, 7 and 8).

The compositions of the present disclosure, and in particular powder formulations, were found to be suitable for addition to foodstuffs and beverages (Example 3 and 10).

Accordingly, the present disclosure advantageously provides a composition of cannabinoids or cannabis-derived compounds in liquid, powder and solid forms that is soluble in water, of natural origin and calorie-free (e.g., less than 5 kcal per serving), that has little or no taste and odor. Further, the compositions of the present disclosure are advantageously both physically and chemically stable, with a high loading of cannabinoids.

Individually and separately these exemplary improvements produce advantageous compositions and dosage forms, and, at times, the combinations of ingredients can provide synergistic beneficial effects on preparation, storage, distribution and/or end use of the compositions. Further improvements are described herein or will become evident from the present disclosure.

Compositions

The present disclosure relates to a composition comprising a cannabinoid or cannabis-derived compound, inulin and pectin.

Cannabis

Cannabis is a genus of flowering plant in the family Cannabaceae. The number of species within the genus is disputed. Three species may be recognized, Cannabis sativa, Cannabis indica and Cannabis ruderalis. C. ruderalis may be included within C. sativa; or all three may be treated as subspecies of a single species, C. sativa. The genus is indigenous to central Asia and the Indian subcontinent.

Cannabis has long been used for hemp fiber, hemp oils, medicinal purposes, and as a recreational drug. Industrial hemp products are made from cannabis plants selected to produce an abundance of fiber. To satisfy the UN Narcotics Convention, some cannabis strains have been bred to produce minimal levels of tetrahydrocannabinol (THC), the principal psychoactive constituent. Many additional plants have been selectively bred to produce a maximum level of THC. Various compounds, including hashish and hash oil, may be extracted from the plant.

Within naturally occurring and manmade hybrids, cannabis contains a vast array of compounds. Three compound classes are of interest within the context of the present disclosure, although other compounds can be present or added to the compositions to optimize the experience of a given recreational consumer and medical or medicinal patient or patient population. Those classes include cannabinoids, terpenes and flavonoids.

There are many ways of growing cannabis, some of which are natural, and some are carefully designed by humans, and they will not be recited here. However, one of ordinary skill in the art of cannabis production will typically place a cannabis seed or cutting into a growth media such as soil, manufactured soil designed for cannabis growth or one of many hydroponic growth media. The cannabis seed or cutting is then provided with water, light and, optionally, a nutrient supplement. At times, the atmosphere and temperature are manipulated to aid in the growth process. Typically, the humidity, air to carbon dioxide gas ratio and elevated temperature, either by use of a heat source or waste heat produced by artificial light, are used. On many occasions ventilation is carefully controlled to maintain the conditions described above within an optimal range to both increase the rate of growth and, optionally, maximize the plant's production of the compounds, which comprise the compositions of the disclosure. It is possible to control lighting cycles to optimize various growth parameters of the plant.

Given the number of variables and the complex interaction of the variables, it is possible to develop highly specific formulas for production of cannabis which lead to a variety of desired plant characteristics. The present disclosure is applicable to use with such inventive means for growing cannabis as well as any of the variety of conventional methods.

Cannabis sativa is an annual herbaceous plant in the Cannabis genus. It is a member of a small, but diverse family of flowering plants of the Cannabaceae family. It has been cultivated throughout recorded history, used as a source of industrial fiber, seed oil, food, recreation, religious and spiritual moods and medicine. Each part of the plant is harvested differently, depending on the purpose of its use. The species was first classified by Carl Linnaeus in 1753.

Cannabis indica, formally known as Cannabis sativa forma indica, is an annual plant in the Cannabaceae family. A putative species of the genus Cannabis.

Cannabis ruderalis is a low-THC species of Cannabis, which is native to Central and Eastern Europe and Russia. It is widely debated as to whether C. ruderalis is a sub-species of Cannabis sativa. Many scholars accept Cannabis ruderalis as its own species due to its unique traits and phenotypes that distinguish it from Cannabis indica and Cannabis sativa.

Cannabis-Derived Compounds

As used herein, the term “cannabis-derived compound” refers to a compound found in a cannabis plant, such as for example a compound that has been obtained and/or extracted from cannabis. The method of conversion typically involves harvesting and, optionally, one of the extraction, fractionation, or purification steps described herein. More typically, a combination of two or more such steps, more typically yet 2, 3, 4, 5, 6, 7, 8, 9, or 10 individual steps described herein. More typically still a combination of separating the cannabis from the media in which it is grown, drying to reduce the water content, grinding to form a power, extraction and, optionally, a fractionation or purification step is performed.

More typically, the process comprises separation of the cannabis-derived compound from the media in which it is grown followed by 2, 3, 4, or 5 steps as described above are performed, more typically yet, 2, 3, or 4 steps are performed.

Suitably, the cannabis-derived compound is separated from the media in which it is grown and first dried and then ground. Once in the ground state, it is, optionally, sieved and finally the resins of the plant are extracted. These resins comprise the cannabis-derived compounds used in the formulations of the disclosure or additional synthetic or semisynthetic compounds may be added to the resins. Remembering that optional fractionation and purification steps are possible, the formulations of the disclosure may have compounds removed from the resin. At that point, again optionally, synthetic or semisynthetic compounds may be added to the resin to form the formulations of the disclosure.

Some steps that can optionally be performed to improve the utility of the compositions include: addition, removal or control of the absolute concentrations of compounds comprising the compositions, direct breeding of cannabis strains, genetic manipulation by methods known in the field of molecular biology such as gene insertion or deletion, lyophilization and the development of polyploid variants by use of compounds such as colicine.

Suitable cannabis-derived compounds include, for example, cannabis concentrate, cannabis extract, cannabis resin, cannabis distillate, cannabis isolate, cannabinoids, terpenes, and combinations thereof.

In an embodiment, the cannabis-derived compound is a cannabinoid.

In an embodiment, the cannabis-derived compound is a terpene.

Cannabinoids

The compositions of the present disclosure comprise a cannabinoid or a cannabis-derived compound. The cannabis-derived compound may be a cannabinoid, or may be an alternative compound derived from cannabis, such as a terpene.

In an embodiment, the compositions comprise a cannabinoid. The compositions may comprise a single cannabinoid (e.g. THC, CBD or another cannabinoid) or may comprise any combination of two or more cannabinoids (e.g. CBD and THC).

As used herein, the term “cannabinoid” refers to a compound belonging to a class of secondary compounds commonly found in plants of genus cannabis, but also encompasses synthetic and semi-synthetic cannabinoids.

In an embodiment, a cannabinoid is one of a class of diverse chemical compounds that acts on cannabinoid receptors such as CB1 and CB2 in cells that alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially as set forth above). The most notable cannabinoid of the phytocannabinoids is tetrahydrocannabinol (THC), the primary psychoactive compound in cannabis. Cannabidiol (CBD) is another cannabinoid that is a major constituent of the plant. There are at least 113 different cannabinoids isolated from cannabis, exhibiting varied effects.

In one embodiment, the cannabinoid is a compound found in a plant, e.g., a plant of genus cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5-diarylpyrazoles, quinolines, and arylsulfonamides as well as eicosanoids related to endocannabinoids.

In many cases, a cannabinoid can be identified because its chemical name will include the text string “*cannabi*”. However, there are a number of cannabinoids that do not use this nomenclature, such as for example those described herein.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-MO-THC), cis-Δ10-tetrahydrocannabinol (cis-Δ10-THC), Tetrahydrocannabinolic acid C4 (THCA-C4), Tetrahydrocannbinol C4 (THC C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor-9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9-Dihydroxy-Δ6a(10a)-tetrahydrocannabinol (8,9-Di-OH-CBT-05), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

In an embodiment, the cannabinoid is a cannabinoid dimer. The cannabinoid may be a dimer of the same cannabinoid (e.g. THC-THC) or different cannabinoids. In an embodiment, the cannabinoid may be a dimer of THC, including for example cannabisol.

As used herein, the term “THC” refers to tetrahydrocannabinol. “THC” refers to and is used interchangeably herein with “Δ9-THC”.

In an embodiment, the cannabinoid is THC (Δ9-THC), Δ8-THC, trans-MO-THC, cis-Δ10-THC, THCA, THCV, Δ8-THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, CBT, or any combination thereof, each having the following exemplary structural formula:

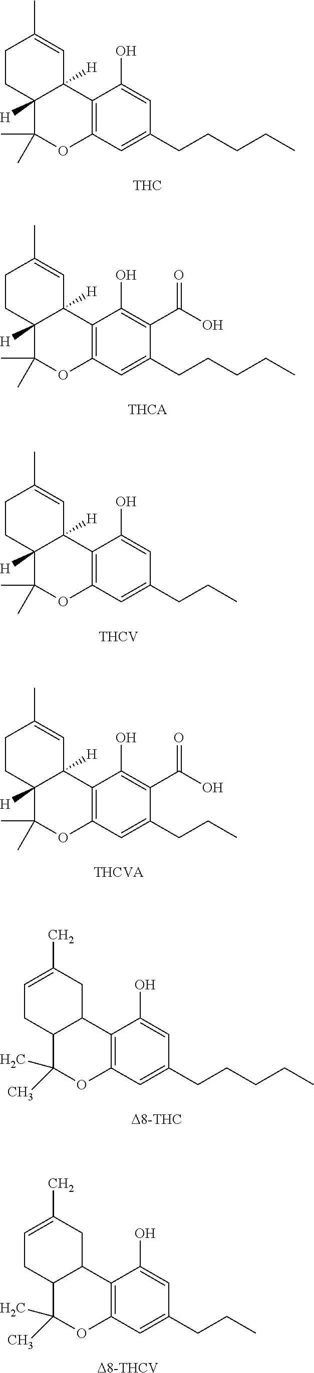

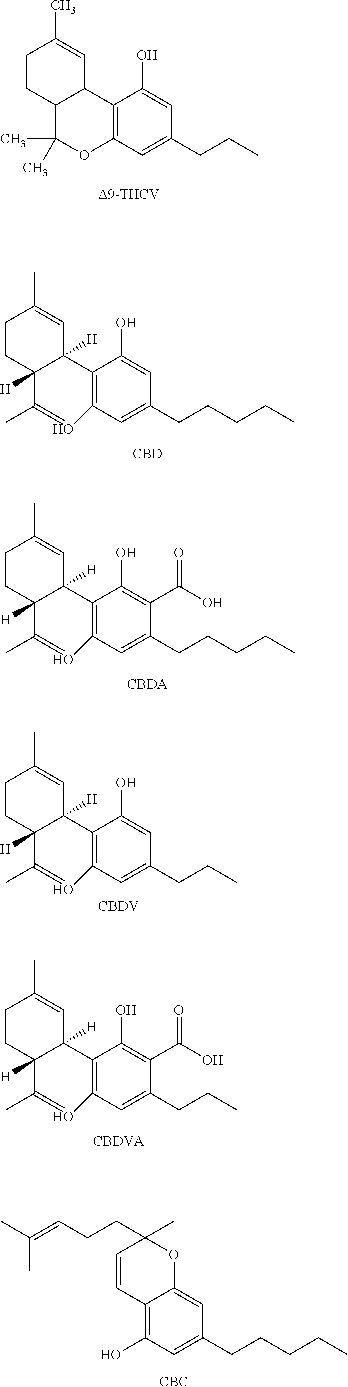

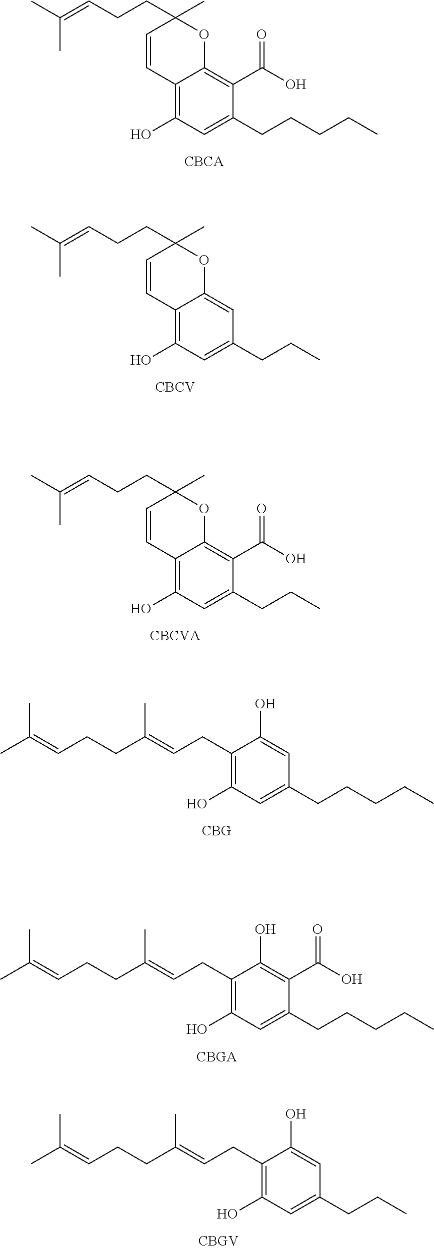

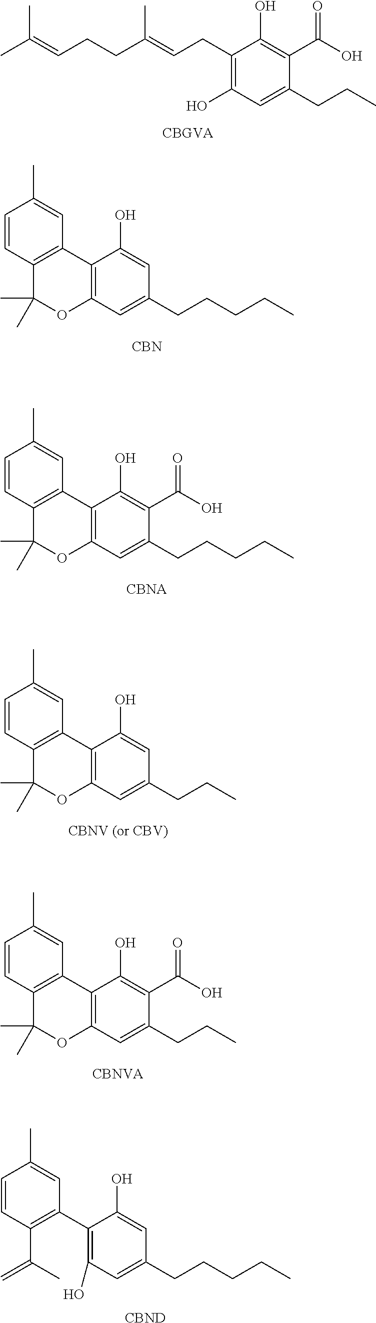

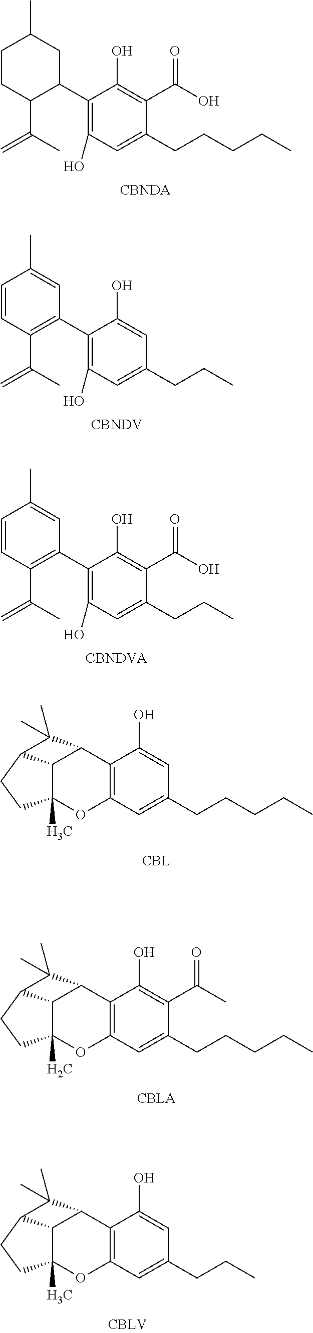

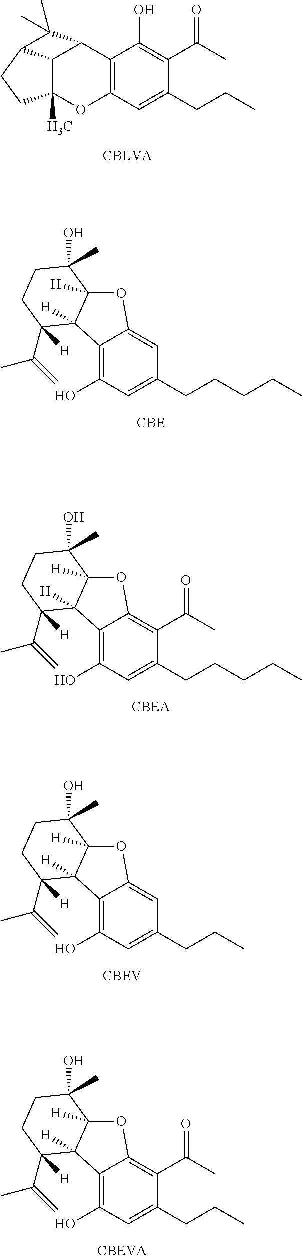

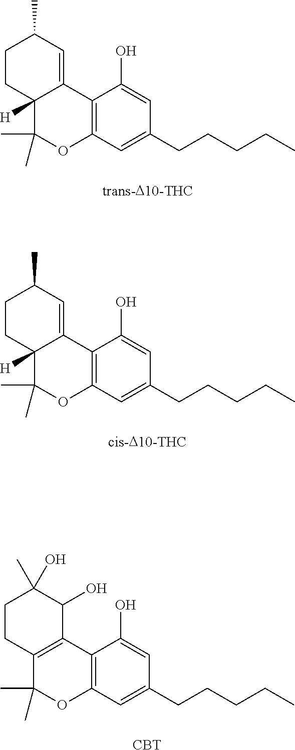

In an embodiment, the cannabinoid is THC, CBD, CBN, CBG, CBGA, or any combination thereof.

Tetrahydrocannabinol (THC) refers to a psychotropic cannabinoid and is the principal psychoactive constituent of cannabis. Its chemical name is (−)-trans-Δ⁹-tetrahydrocannabinol.

Cannabidiol (CBD) is one of the active cannabinoids identified in cannabis. It is a major phytocannabinoid, by some accounts making up to 40% of the plant's extract. CBD does not appear to have any intoxicating effects such as those caused by THC in marijuana, but may have effects on anxiety, depression and have an anti-psychotic effect, and have effects on other comorbidities. In some instances, the comorbidities are related to disorders described herein such as pain and post-traumatic stress disorders commonly referred to as “PTSD.”

Cannabinol (CBN) is thought to be a non-psychoactive cannabinoid found only in trace amounts in Cannabis and can be produced via oxidative degradation of THCA and THC. Pharmacologically relevant quantities are formed as a metabolite of tetrahydrocannabinol (THC). CBN acts as a partial agonist at the CB1 receptors, but has a higher affinity to CB2 receptors, however; with lower affinities in comparison to THC. Degraded or oxidized cannabis products, such as low-quality baled cannabis and traditionally produced hashish, are high in CBN, but modern production processes have been alleged to minimize the formation of CBN. Cannabinol has been shown to have analgesic properties. Unlike other cannabinoids, CBN does not stem from cannabigerol (CBG).

Cannabigerol (CBG) is thought to be a non-intoxicating cannabinoid found in the Cannabis genus of plants. CBG is the non-acidic form of cannabigerolic acid (CBGA), the parent molecule (“mother cannabinoid”) from which many other cannabinoids are obtained. CBG has been found to act as a high affinity α2-adrenergic receptor agonist, moderate affinity 5-HT1A receptor antagonist, and low affinity CB1 receptor antagonist. It also binds to the CB2 receptor as an antagonist.

Cannabigerolic Acid (CBGA or CBG-A) is the alleged primordial phyto-cannabinoid. It is the alleged compound in cannabis from which all the plant's other naturally occurring cannabinoids are formed; without CBGA, the cannabis plant cannot produce its most useful compounds.

In an embodiment, the cannabinoid is THC (Δ9-THC), Δ8-THC, trans-MO-THC, cis-Δ10-THC, CBD, CBC, CBG, CBL, CBN, CBT, or any combination thereof.

In an embodiment, the cannabinoid is THC or CBD, or a combination thereof.

In an embodiment, the cannabinoid is THC.

In an embodiment, the cannabinoid is CBD.

In particularly suitable embodiments, the cannabinoid includes one or more cannabinoids, and in particular, a combination of THC and CBD. Particularly, the liquid formulation may include about 10 mg/mL cannabinoids. Likewise, the powder formulation may include about 10 mg/g cannabinoids. A bulking agent may be used to dilute the powder to the requisite dosage of cannabinoids.

As noted above, in some embodiments, the cannabinoid includes a combination of THC and CBD. In exemplary embodiments, the compositions can include THC and CBD in weight ratios of THC:CBD of from about 100:0.1 to about 0.1:100; including about 1:1.

In select embodiments of the compositions disclosed herein, the cannabinoids may be introduced in the form of pure cannabinoids or as a cannabis concentrate. As used herein, “pure cannabinoids” is meant to refer to a single cannabinoid or a mixture of different cannabinoids that is free of other compounds. The pure cannabinoids may be contained in solution in a diluent or other medium, or may be a liquid or solid form of the pure cannabinoids absent any diluent. In an embodiment, the pure cannabinoids are synthetic or semi-synthetic cannabinoids. As used herein, “cannabis concentrate” is meant to refer a concentrated composition of cannabinoids, such a cannabinoid extract from a plant. Non-limiting exemplary embodiments of a cannabis concentrate include a cannabis distillate, a cannabis isolate, a cannabis oil, or any other type of extract containing one or more cannabinoids. In an embodiment, the cannabis concentrate is a cannabis distillate or isolate dissolved in a carrier solvent described herein, such as for example coconut oil or MCT oil.

As described in greater detail elsewhere herein, in addition to cannabinoids, the compositions of the present disclosure may also include additives, such as for example terpenes, terpenoids, flavonoids, and the like and combinations thereof.

In an embodiment, the additives (e.g. terpenes and/or flavonoids) are independently or in combination derived from natural sources and are selected to be stable in the selected compositions, dosage forms, beverages or foodstuffs herein. More suitably still, in some embodiments, the composition or beverage of the present disclosure with additives is clear, stable at room temperature and capable of being provided in both bulk and unit dose forms. More suitably yet, in some embodiments, the additives may act synergistically in the compositions to provide desirable production, storage, distribution or end use.

Another suitable embodiment of the compositions, dosage forms, beverages or foodstuffs of the present disclosure provides fast onset of biological effects of the cannabinoids in human or animal consumers or subjects.

Inulin and Pectin

The compositions of the present disclosure comprise inulin and pectin. The skilled person will know what each of these components comprise, and would know whether any particular compound, mixture, or extract is or comprises inulin or pectin. The methods and/or analysis used to confirm the presence of inulin or pectin are known to the skilled person. In this regard, the following description of the chemical structure/composition of inulin and pectin is intended to be illustrative and not limiting.

Inulin (C₆ₙH₁₀ₙ₊₂O₅ₙ₊₁) is a heterogeneous mixture of fructose polymers typically extracted from chicory. Other exemplary natural sources of inulin include asparagus, garlic, artichoke, jicama, onions, and yacon root. Inulin consists of a chain-terminating glucose moiety and repetitive fructosyl moieties, which are linked by β-(2→1) bonds. The degree of polymerization for inulin ranges from 2-60.

Inulin may be obtained from any number of plant sources, and may also be modified or manufactured. Oligofructose is a subgroup of inulin made by removing the longer molecules. Typically, oiligofructose comprises fructose molecules of between two to ten units. High-performance (HP) inulin is a subgroup of inulin made by removing the shorter molecules. Typically, HP inulin comprises fructose molecules of between 11 to 60 units, with an average degree of polymerization typically around 25. Fructooligosaccharides (FOS) is a subgroup of inulin consisting of short inulin molecules synthesized from table sugar.

Inulin is not digested in the upper gastrointestinal tract, and therefore has a low caloric value.

The manufacturing process for inulin is rather similar to that of sugar extracted from natural plant sources. The plant material is typically harvested, sliced and washed. Inulin is then extracted from the plant material by using a hot water diffusion process, then purified and dried.

Any form of inulin, including without limitation the subgroups described herein, may be used in the compositions of the present disclosure. In an embodiment, the compositions of the present disclosure comprise inulin having a degree of polymerization typically in the range of 10-60. In an embodiment, the compositions of the present disclosure comprise inulin having a degree of polymerization of about 10 on average.

Pectin, also commonly known as pectic polysaccharides, is a complex heteropolysaccharide comprising a mixture of galacturonic acid-rich polysaccharides. Pectin is commonly found in the primary cell walls of plants. While the exact chemical structure or composition of pectin is still under debate and may vary depending on source (e.g. citrus versus legume), the two main polysaccharides in pectin are homogalacuronans (HG) and rhamnogalacturonan-I (RG-I).

HGs are linear chains of α-(1→4)-linked D-galacturonic acid, each of which contains a carboxylic group. HGs typically comprise approximately 65% of the pectin structure. HG content confers pectins hydrophilic character as well as increasing its ability to form a gel in the presence of divalent ions such as calcium. HG also promotes the formulation of large oil droplets in emulsions and lowers emulsion capacity and stability. RG-I are pectic polysaccharides that have a repetitive disaccharide backbone composed of D-galacturonic acid and L-rhamnose. RG-I typically comprises approximately 20-35% of the pectin structure.

The majority of carboxyl groups in pectin are often esterified with methoxy groups. Pectin from some sources may also be heavily acetylated. The presence of acetyl ester groups lowers the hydrophilic character of polysaccharide and increases both emulsion capacity and stability. Pectin may also contain neutral side chains branched off the main polysaccharide chain. The sugars on these neutral side chains typically contain little to no carboxylic acid groups and have been shown in certain instances to increase emulsion stability, likely through interactions with the protein moiety of pectin. Esterified ferulic acid groups may also be present in the pectin, which confer a significant hydrophobic character to pectin as well as providing a potential site for non-toxic covalent crosslinking.^{[1]} The protein moiety of pectin may be an important feature in terms of its emulsification potential. The protein content of pectin has been shown to bind preferentially to the surface of oil droplets in emulsions, and the removal of pectin protein content via proteases removes most of its emulsification potential.^{[2]}

In an embodiment of the present disclosure, the D-galacturonic acid content of pectin ranges from 60-80% w/w and the percentage of methyl-esterified D-galacturonic acid residues ranges 1-80% w/w.

Pectin may be obtained from any number of different sources. Pears, apples, guavas, quince, plums, gooseberries, and oranges and other citrus fruits typically contain large amounts of pectin, while soft fruits, like cherries, grapes, and strawberries, typically contain small amounts of pectin. Some plants, such as sugar beet, potatoes and pears, contain pectins with acetylated galacturonic acid in addition to methyl esters.

From a source material, pectin is typically extracted by adding hot dilute acid at pH-values from 1.5-3.5. During several hours of extraction, the protopectin loses some of its branching and chain length and goes into solution. After filtering, the extract is concentrated in a vacuum and the pectin is then precipitated by adding ethanol or isopropanol.

Citrus peel pectin is commonly used as a gelling agent in the food industry, but also has significant potential as an emulsifier. Sugar beet pectin, while less commonly used, is also an effective emulsifying agent.^{[3]} Sugar beet pectin typically contains a lower HG content and many times more neutral side chains than citrus pectin, making it less hydrophilic than citrus peel pectin and increasing its potential to produce small and stable oil droplets in an emulsion. These differences in polysaccharide content also mean that sugar beet pectin is less likely to gel and/or crosslink with calcium ions to the same degree as citrus peel pectin. Sugar beet pectin also contains more acetylated carboxyl groups and about 10 times more feruloyl esters than citrus peel pectin, significantly increasing its hydrophobic character and potential for covalent crosslinking. The protein content is also greatly increased in sugar beet pectin as compared to citrus peel pectin.

As shown herein, sugar beet pectin may represent an advantageous pectin for particular types of beverages and foodstuffs due to improved encapsulation efficiency and improved solubility in water (see Example 11). Key characteristics of citrus peel and sugar beet pectin are shown in Table 1 below.

TABLE 1

| | Citrus peel pectin | Sugar beet pectin |
|---|---|---|
| HG % | 77%^{[4]} | 29%^{[5]} |
| (w/w % of total | | |
| polysaccharide content) | | |
| Neutral Side Chains % | 4%^{[4]} | 48%^{[5]} |
| (w/w % of total | | |
| polysaccharide content) | | |
| Acetyl Content | ~1.5%^{[6]} | 3-5%^{[7]} |
| (w/w % of dry mass) | | |
| Feruloyl Ester Content | ~0.10%^{[6]} | 1.0%^{[7]} |
| (w/w % of dry mass) | | |
| Protein Content | <1.0%^{[6]} | 2-3%^{[7]} |
| (w/w % of dry mass) | | |

In an embodiment, the compositions of the present disclosure comprise a pectin obtained or extracted from a natural source. In an embodiment, the pectin is from a single source. For example, in select embodiments, the pectin is a citrus pectin (or citrus peel pectin) obtained or extracted from a citrus fruit, such as oranges. In other select embodiments, the pectin is a sugar beet pectin obtained or extracted sugar beet plant. In other embodiments, the pectin may be a mixture of pectins obtained from different sources.

Alternatively, the pectin may be derived synthetically, for example to mimic the properties of a pectin obtained or extracted from a natural source. In other embodiments still, the pectin may be obtained or extracted from one or more natural sources, but subsequently modified. For example, in an embodiment the pectin is modified citrus pectin (MCP).

The compositions herein may comprise any suitable concentration of inulin and pectin. In an embodiment, the compositions herein comprise a ratio of inulin:pectin of between about 99%:1% w/w to about 60%:40% w/w. In select embodiments, the ratio of inulin:pectin is between about 95%:5% w/w to about 60%:40% w/w. In select embodiments, the ratio of inulin:pectin is between about 95%:5% w/w to about 70%:30% w/w. In select embodiments, the ratio of inulin:pectin is between about 95%:5% w/w to about 80%:20% w/w. In select embodiments, the ratio of inulin:pectin is about 95%:5% w/w, about 90%:10% w/w, about 85%:15% w/w, about 80%:20% w/w, about 75%:25% w/w, about 70%:30% w/w, about 65%:35% w/w, or about 60%:40% w/w.

In a particular embodiment, the ratio of inulin:pectin is about 95%:5% w/w. In a particular embodiment, the ratio of inulin:pectin is about 80%:20% w/w. In a particular embodiment, the ratio of inulin:pectin is about 70%:30% w/w. In a particular embodiment, the ratio of inulin:pectin is about 77.5%:22.5% and the pectin is sugar beet pectin.

The compositions herein may comprise any suitable combined concentration of inulin and pectin, relative to other components in the composition. In an embodiment, the compositions herein comprise between about 50% w/w to about 90% w/w of inulin and pectin relative to the total mass of the composition. In an embodiment, the compositions herein comprise about 50% w/w, about 55% w/w, about 60% w/w, about 65% w/w, about 70% w/w, about 75% w/w, about 80% w/w, about 85% w/w, or about 90% w/w of inulin and pectin relative to the total mass of the composition.

The compositions herein may also comprise any suitable combined concentration of inulin and pectin, relative to the by weight amount of cannabinoid, cannabis-derived compound or cannabis concentrate. In an embodiment, the compositions herein comprise between about 50% w/w to about 90% w/w of inulin and pectin relative to the total mass of cannabinoid, cannabis-derived compound or cannabis concentrate. In an embodiment, the compositions herein comprise about 50% w/w, about 55% w/w, about 60% w/w, about 65% w/w, about 70% w/w, about 75% w/w, about 80% w/w, about 85% w/w, or about 90% w/w of inulin and pectin relative to the total mass of cannabinoid, cannabis-derived compound or cannabis concentrate.

Additives

In some embodiments, the compositions of the present disclosure may include additives, such as for example and without limitation terpenes, terpenoids, flavonoids, or any combination thereof.

In an embodiment, the additives may be derived from cannabis plants. In an embodiment, the additives may be derived from natural sources other than a cannabis plant, such as a plant of a different species. Alternatively, in some embodiments, the additives may be synthetic or semi-synthetic compounds.

Terpenes and Terpenoids

In an embodiment, the compositions herein may comprise one or more terpenes and/or terpenoids.

Within the context of this disclosure, the term “terpene” includes cannabis derived terpenes and non-cannabis derived terpenes.

Terpenes are a large and diverse class of organic compounds, produced by a variety of plants, particularly conifers, and by some insects such as termites or swallowtail butterflies, which emit terpenes from their osmetieria. Terpenes are also major constituents of Cannabis sativa plants. They often have a strong odor and may protect the plants that produce them by deterring herbivores and by attracting predators and parasites of herbivores. The difference between terpenes and terpenoids is that terpenes are hydrocarbons, whereas terpenoids contain additional functional groups.

They are the major components of resin, and of turpentine produced from resin. The name “terpene” is derived from the word “turpentine”. In addition to their roles as end-products in many organisms, terpenes are major biosynthetic building blocks within nearly every living creature. Steroids, for example, are derivatives of the triterpene squalene.

When terpenes are modified chemically, such as by oxidation or rearrangement of the carbon skeleton, the resulting compounds are generally referred to as terpenoids. Some authors will use the term terpene to include all terpenoids. Terpenoids are also known as isoprenoids.

Within the context of this disclosure, the term “terpene” includes hemiterpenes, monoterpenols, terpene esters, diterpenes, monoterpenes, polyterpenes, tetraterpenes, terpenoid oxides, sesterterpenes, sesquiterpenes, norisoprenoids, or their derivatives. As well as isomeric, enantiomeric, or optically active derivatives.

Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids. These derivatives are encompassed herein by the term “terpene”, unless specifically stated otherwise.

Within the context of this disclosure, the term terpene includes the α-(alpha), β-(beta), γ-(gamma), oxo-, isomers, or any combinations thereof.

Terpenes are the primary constituents of the essential oils of many types of plants and flowers. Essential oils are used widely as fragrances in perfumery, and in medicine and alternative medicines such as aromatherapy. Synthetic variations and derivatives of natural terpenes also greatly expand the variety of aromas used in perfumery and flavors used in food additives.

Higher amounts of terpenes are released by trees in warmer weather, acting as a natural form of cloud seeding. The clouds reflect sunlight, allowing the forest to regulate its temperature. The aroma and flavor of hops comes, in part, from sesquiterpenes (mainly alpha-humulene and beta-caryophyllene), which affect beer quality. Accordingly, in some embodiments, the compositions of the present disclosure include hop-derived terpenes such as hop-derived terpene blends available as Aramis, Brewer's Gold, Bravo and the like, and combinations thereof.

Plant terpenes are used extensively for their aromatic qualities and play a role in traditional herbal remedies. Terpenes contribute to the scent of eucalyptus, the flavors of cinnamon, cloves, and ginger, the yellow colour in sunflowers, and the red colour in tomatoes.

Non-limiting examples of terpenes within the context of this disclosure include: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

In select embodiments, the compositions disclosed herein comprise a terpene selected from β-caryophyllene, caryophyllene oxide, borneol, 1,8-cineole, camphene, humulene (e.g., α-humulene), limonene (e.g., D-limonene, L-limonene), linalool, myrcene (e.g., β-myrcene), nerolidol, pulegone, α-pinene, β-pinene, para-cymene, eugenol, farnesol, geraniol, phytol, terpineol (e.g., α-terpineol) and terpinolene, or any combination thereof.

In particularly suitable embodiments, the compositions of the present disclosure may comprise terpenes having antimicrobial properties. Exemplary antimicrobial terpenes include, for example, Ocimum basilicum (basil), Laurus nobilis (bay), Cinnamomum verum (Ceylon cinnamon), Capsicum annuum (paprika), Syzygium aromaticum (clove), Mentha piperita (peppermint), Tanacetum vulgare (tansy), Artemisia dracunculus (Tarragon), and the like as known in the art.

Flavonoids

In some embodiments, the compositions of the present disclosure may include additives such as one or more flavonoids.

As used herein, the term “flavonoid” refers to any compound of a large class of plant pigments having a structure based on or similar to that of flavone. Chemically, flavonoids have the general structure of a 15-carbon skeleton, which consists of two phenyl rings and a heterocyclic ring.

Within the context of this disclosure, the term “flavonoids” includes bioflavonoids, isoflavonoids and neoflavonoids. Isoflavones use the 3-phenylchromen-4-one skeleton (with no hydroxyl group substitution on carbon at position 2). Examples include: Genistein, Daidzein, Glycitein, Isoflavanes, Isoflavandiols, Isoflavenes, Coumestans, and Pterocarpans.

Within the context of this disclosure, the term “flavonoids” also includes anthocyanidins, anthoxanthins, flavanones, flavanonols and flavens.

Flavonoids are widely distributed in plants, fulfilling many functions. Flavonoids are the most important plant pigments for flower colouration, producing yellow or red/blue pigmentation in petals designed to attract pollinator animals. In higher plants, flavonoids are involved in UV filtration, symbiotic nitrogen fixation and floral pigmentation. They may also act as chemical messengers, physiological regulators, and cell cycle inhibitors. Some flavonoids have inhibitory activity against organisms that cause plant diseases, e.g. Fusarium oxysporum.

Sources of flavonoids include, without limitation, cannabis, parsley, blueberries, black tea, citrus, wine, cocoa and peanut.

Additional exemplary flavonoids include Apigenin, beta-sitosterol, cannaflavin A, kaempferol, luteolin, orientin, and quercetin.

In an embodiment, the flavonoid is cannaflavin.

Other Additives

The compositions of the present disclosure may include any number of other additives, including without limitation a solvent, a carrier solvent, a bulking agent, an antioxidant, a viscosity modifying agent, a nutritional supplement, or a stabilizer. These components may be used either alone or in combination to improve, for example, the chemical and/or physical properties, stability, nutritional profile, taste, colour and/or viscosity, of the compositions disclosed herein or a beverage or foodstuff produced therefrom. In this regard, certain additives (e.g. stabilizers) may be added to the beverage or foodstuff separately from the compositions disclosed herein.

In an embodiment, the compositions herein are a liquid formulation and comprise a solvent. As used herein, “solvent” is intended to refer to the medium that constitutes the continuous phase of the liquid formulation. In contrast, “a carrier solvent” is intended to refer to a solvent in which the cannabinoids or cannabis-derived compounds are dissolved and is a discontinuous phase of the liquid formulations herein. In an embodiment, the solvent of the liquid formulations of the present disclosure is an aqueous solvent. In select embodiments, the solvent is water, a saline solution, a phosphate buffered saline, or other aqueous solution. In a particular embodiment, the solvent is water. As described elsewhere herein, in an embodiment the liquid formulations of the present disclosure are an emulsion.

In an embodiment, the compositions herein comprise a carrier solvent. The carrier solvent may be any suitable solvent capable of mixing with, or dissolving, the cannabinoid or cannabis-derived compound. In an embodiment, the carrier solvent is an oily medium. By “oily medium” it is meant to refer to a medium capable of dissolving lipophilic or hydrophobic compounds, such as cannabinoids.

A non-limiting list of exemplary carrier solvents includes ethanol, isopropanol, dimethyl sulfoxide, acetone, ethyl acetate, pentane, heptane, diethyl ether, medium-chain triglycerides (MCT oil), medium-chain fatty acids (e.g., caproic acid, caprylic acid, capric acid, lauric acid), long-chain triglycerides (LCT oil), long-chain fatty acids (e.g., myristic acid, palmitic acid, stearic acid, arachidic acid, linoleic acid), glycerine/glycerol, monoglycerides (e.g. glyceryl monostearate, glyceryl hydroxystearate, glyceryl monoleate, winterized glyceryl monoleate, monolaurin, glyceryl monolinoleate, Maisine® CC, Peceol™), coconut oil, corn oil, canola oil, olive oil, avocado oil, vegetable oil, flaxseed oil, palm oil, palm kernel oil, peanut oil, sunflower oil, rice bran oil, safflower oil, jojoba oil, argan oil, grapeseed oil, castor oil, wheat germ oil, peppermint oil, hemp oil, sesame oil, terpenes, terpenoids, β-myrcene, linalool, α-pinene, β-pinene, β-caryophyllene, caryophyllene oxide, α-humulene, nerolidol, D-limonene, L-limonene, para-cymene, eugenol, farnesol, geraniol, phytol, menthol, terpineol, α-terpineol, benzaldehyde, hexyl acetate, methyl salicylate, eucalyptol, ocimene, terpinolene, α-terpinene, isopulegol, guaiol, α-bisabolol and combinations thereof. Other suitable carrier solvents include Labrasol, Labrafac Lipophile WL 1349, Labrafil M1944, Peceol, Plurol Oliqiue CC 497, Transcutol HP, Tween 80, Gelucire 48/16, Vitamin E TPGS, and combinations thereof.

In an embodiment, the carrier solvent may be combined with the cannabinoid or cannabis-derived compound prior to mixing the cannabinoid or the cannabis-derived compound with the inulin and pectin. In an embodiment, the carrier solvent may be present with the cannabinoid or cannabis-derived compound in the inner hydrophobic core of the composition compositions disclosed herein.

In select embodiments, the carrier solvent may comprise coconut oil. In select embodiments, the carrier solvent may comprise medium-chain triglyceride (MCT) oil. As used herein, MCTs are triglycerides with two or three fatty acids having an aliphatic tail of 6-12 carbon atoms, i.e., medium-chain fatty acids (MCFAs). Sources rich in MCTs for commercial extraction of MCTs include palm kernel oil and coconut oil.

In an embodiment of the compositions disclosed herein, the weight ratio of the carrier solvent to the cannabinoid or cannabis-derived compound may be from about 3:1 to about 1:3. In select embodiments, the weight ratio of the carrier solvent to the cannabinoid or cannabis-derived compound may be from about 2:1 to about 1:2. In a particular embodiment, the weight ratio of carrier solvent to the cannabinoid or cannabis-derived compound is about 1:1. Where additional additives are included in the compositions, such as terpenes, terpenoids, flavonoids or nutritional supplements, it is intended that these additives are included with the cannabinoid or cannabis-derived compound in the calculation of the weight ratio. For example, a composition may have a hydrophobic inner core comprising 50% w/w of a carrier solvent and 50% w/w of a combination of cannabinoids, terpenes, terpenoids, flavonoids and/or nutritional supplements as a 1:1 w/w ratio.

In some embodiments, the powder formulation of the present disclosure can be diluted with a bulking agent or a mixture of bulking agents. For example, a bulking agent may be used to dilute the cannabinoid dosage in a powder composition to the requisite amount. Suitable bulking agents include, for example, gum arabic, waxy maize starch, dextrin, maltodextrin, polydextrose, inulin, fructooligosaccharide, sucrose, glucose, fructose, galactose, lactose, maltose, trehalose, cellobiose, lactulose, ribose, arabinose, xylose, lyxose, allose, altrose, mannose, gulose, talose, erythritol, threitol, arabitol, xylitol, mannitol, ribitol, galactitol, fucitol, inositol, maltitol, sorbitol, isomalt, lactitol, polyglycitol, iditol, volemitol, maltotriitol, maltotetraitol, maltol, myo-inositol, stevia, stevio side, rebaudio side, neotame, sucralose, saccharin, sodium cyclamate, aspartame, acesulfame potassium, chitin, and chitosan. In an embodiment, the bulking agent is erythritol. In an embodiment, the bulking agent is sucrose. In an embodiment, the bulking agent is inositol. In an embodiment, the bulking agent is a BioSteel™ sports drink powder.

In some embodiments, the bulking agent comprises myo-inositol. Myo-inositol, or (1R,2S,3r,4R,5S,6s)-cyclohexane-1,2,3,4,5,6-hexol, is a sugar alcohol with half the sweetness of sucrose (table sugar). It is made naturally in humans from glucose. Myo-inositol is a particularly suitable bulking agent for the compositions of the present disclosure and has been found to improve dissolution of the powder compositions in water (see Example 8).

The method of mixing the bulking agent with the powder composition may comprise any known method, including but not limited to, manual shaking, use of a V-blender, mortar and pestle, and magic bullet blending. In some embodiments, a V-blender is used to mix the bulking agent with the powder composition.

In some aspects, the bulking material may comprise a sweetener, pH modifier, pH stabilizer, antimicrobial preservative, antioxidant, texture modifier, colourant or combinations thereof.

In some embodiments, the bulked powder formulations comprise at least 0.001% by weight, and suitable from 0.001% by weight to about 3% by weight, of a cannabinoid or a cannabis-derived compound. More suitably, a composition for an exemplary product may include 10 milligram of tetrahydrocannabinol (THC) per serving. Assuming a 3.5 gram serving size, the bulk powder formulation would contain approximately 0.3% by weight of the primary cannabinoid (e.g. THC and/or CBD). Assuming a 5 gram sample size, the bulk powder formulation would contain approximately 0.2% by weight of the primary cannabinoid.

In some embodiments, the compositions disclosed herein may comprise a nutritional supplement. Nutritional supplements comprise substances useful to the consumer of the compositions disclosed herein, or beverages or foodstuffs prepared therewith, for maintenance of normal body health. Suitable nutritional supplements may comprise, for example, essential nutrients including vitamins, dietary minerals, amino acids and fatty acids. Exemplary nutritional supplements may include vitamin A, vitamin B1, vitamin B2, vitamin B3, vitamin B5, vitamin B6, vitamin B7, vitamin B9, vitamin B12, vitamin C, vitamin D, vitamin E, vitamin K calcium, phosphorus, potassium, sulfur, sodium, chlorine, magnesium, iron, cobalt, copper, zinc, molybdenum, iodine, selenium, manganese, nickel, chromium, fluorine, boron, strontium histidine, isoleucine, leucine, lysine, methionine, cysteine, phenylalanine, tyrosine, threonine, tryptophan, valine, alpha-linoleic acid, and linoleic acid.

In some embodiments, the compositions disclosed herein may comprise a viscosity modifier. Viscosity modifiers include any compound or agent capable of altering the viscosity of the compositions disclosed herein, or a beverage or foodstuff produced therewith. Exemplary embodiments of viscosity modifiers include anticaking agents, antifoaming agents, bulking agents, coagulation agents, gelling agents, glazing agents, humectants, leavening agents, tenderizers, and thickeners. In an embodiment, the viscosity modifying agent may be an unmodified starch, pregelatinized starch, cross-linked starches, gums (e.g. guar gum, xanthum gum, acacia), polyvinyl pyrrolidone (PVP), polyethylene oxide, waxes (e.g. beeswax), and mixtures thereof.

Other optional additives include any one or more of acids, bases, acidity regulators, alcohol, antioxidants, food colouring, colour retention agents, emulsifiers, flavor enhancers, flour treatment agents, tracer gases, preservatives, stabilizers, and sweeteners. In an embodiment, the antioxidant may be acorbyl palmitate or α-tocopherol.

In select embodiments, the compositions herein may comprise a stabilizer. In other embodiments, the compositions herein may be used in combination with a stabilizer in preparing a beverage or a food stuff.

As used herein, a stabilizer is any substance used to prevent an unwanted change in state. The stabilizer may be used to improve or maintain the stability of the compositions or to improve or maintain the stability of the end-product (e.g. beverage or foodstuff). For example, cannabinoids or cannabis-derived compounds within the compositions may be susceptible to degradation, such as oxidative degradation.

Non-limiting examples of stabilizers include hydrocolloids (such as alginate, agar, carrageenan, cellulose and cellulose derivatives, gelatin, guar gum, gum Arabic, locust bean gum, pectin, starch and xanthan gum), antioxidants (water-soluble and/or oil-soluble), and chelating agents. Non-limiting examples of chelating agents include: aminopolycarboxylic acids including ethylenediaminetetraacetic acid (EDTA) and its various salts, calixarenes, porphyrins, bipyridines, citric acid, iminodisuccinic acid, and polyaspartic acid. In an embodiment, the compositions of the present disclosure are used in combination with a chelating agent as a stabilizer. In an embodiment, the chelating agent is EDTA.

One other class of common additive or modifier useful in the compositions disclosed herein is the group of substances referred to as phospholipids. In an embodiment, the carrier solvent may comprise phospholipids. In other embodiments, phospholipids may be included as an additive for purposes other than as a carrier solvent.

Phospholipids are made up of two fatty acid tails and a phosphate group head. Fatty acids are long chains mostly made up of hydrogen and carbon, while phosphate groups consist of a phosphorus molecule with four oxygen molecules attached. These two components of the phospholipid are connected via a third molecule, glycerol.

Phospholipids can act as emulsifiers, enabling oils to form a colloid with water. Phospholipids are one of the components of lecithin, which is found in egg-yolks, as well as being extracted from soy beans, and is used as a food additive in many products, and can be purchased as a dietary supplement. Lysolecithins are typically used for water-oil emulsions like margarine, due to their higher HLB ratio.

In one embodiment of the present disclosure, phospholipids are included as additives or modifiers of the compositions disclosed herein. Typically, such compositions are liquids capable of being beverages. Beverages of this type commonly use phospholipid additives or modifiers to solubilize one or more hydrophobic components of the cannabis or cannabis concentrate (e.g. cannabinoids). The methods of solubilization are described herein, such as in respect of carrier solvents. The phospholipids are typically derived from natural sources such as naturally occurring oils from a plant such as coconut, safflower and sunflower. These phospholipids can include secondary products obtained therefrom such as lecithin from sunflower oil. In select embodiments, the phospholipid or derivative therefrom is present in the compositions disclosed herein in amounts of less than 20 weight or volume percent, and suitable, about 0.01-10 weight or volume percent. More typically, 0.01, 0.1, 1 or 10 weight or volume percent, more typically yet 0.1 to 1 weight or volume percent.

In the same fashion as phospholipids, triglycerides are a typical additive or modifier that may be used in the compositions disclosed herein. Triglycerides are chemically tri-esters of fatty acids and glycerol. Triglycerides are formed by combining glycerol with three fatty acid molecules. Alcohols have a hydroxyl (—OH) group. Organic acids have a carboxyl (—COOH) group. Alcohols and organic acids join to form esters. The glycerol molecule has three hydroxyl (—OH) groups. Each fatty acid has a carboxyl group (—COOH). In triglycerides, the hydroxyl groups of the glycerol join the carboxyl groups of the fatty acid to form ester bonds:

HOCH₂CH(OH)CH₂OH+RCO₂H+R′CO₂H+R″CO₂H→RCO₂CH₂CH(O₂CR′)CH₂CO₂R″+3H₂O

The three fatty acids (RCO₂H, R′CO₂H, R″CO₂H in the above equation) are usually different, but many kinds of triglycerides are known. The chain lengths of the fatty acids in naturally occurring triglycerides vary, but most contain 16, 18, or 20 carbon atoms. Natural fatty acids found in plants and animals are typically composed of only even numbers of carbon atoms, reflecting the pathway for their biosynthesis from the two-carbon building-block acetyl CoA. Bacteria, however, possess the ability to synthesize odd- and branched-chain fatty acids. As a result, ruminant animal fat contains odd-numbered fatty acids, such as 15, due to the action of bacteria in the rumen. Many fatty acids are unsaturated, some are polyunsaturated (e.g., those derived from linoleic acid).

Most natural fats contain a complex mixture of individual triglycerides. Because of this, they melt over a broad range of temperatures. Cocoa butter is unusual in that it is composed of only a few triglycerides, derived from palmitic, oleic, and stearic acids in the 1-, 2-, and 3-positions of glycerol, respectively.

In embodiments wherein triglycerides are used as additives or modifiers of the compositions disclosed herein, they may be present in about 0.01-10 weight or volume percent. More typically, 0.01, 0.1, 1 or 10 weight or volume percent, more typically yet 0.1 to 1 weight or volume percent.

Natural phospholipid derivatives include egg PC (Egg lecithin), egg PG, soy PC, hydrogenated soy PC, and sphingomyelin. Synthetic phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), PEG phospholipid (mPEG-phospholipid, polyglycerin-phospholipid, functionalized-phospholipid, and terminal activated-phospholipid).

Phospholipids can form cell, micelle and liposomal membranes as well as other self-organizing multi-molecular structures because the phosphate group head is hydrophilic (water-loving) while the fatty acid tails are hydrophobic (water-hating). They automatically arrange themselves in a certain pattern in water or other polar environment because of these properties, and form membranes. To form membranes, phospholipids line up next to each other with their heads on the outside of the polar medium and their tails on the inside, thus forming an inner and outer surface. A second layer of phospholipids also forms with heads facing the inside of the structure and tails facing away. In this way, a double layer is formed with phosphate group heads on the outside, and fatty acid tails on the inside. This double layer, called a lipid bilayer, forms the main part of the membrane or other similar structure.

Core-Shell Structures

The compositions of the present disclosure comprise compounds that are lipophilic (e.g. cannabinoids) and typically have low solubility in hydrophilic solutions or substances (e.g., aqueous solutions used for preparing conventional beverages and foods). One method for obtaining desirable compositions comprising these lipophilic compounds in hydrophilic solutions or substances is to encapsulate or disperse the lipophilic substances in the hydrophilic solution or substance using inulin and pectin as described herein, which provide an environment for stable oil-in-water emulsions, micelles, liposomes or other complex phase equilibrium modified compositions.

An exemplary method of preparing a stable oil-in-water composition is to use an emulsion (e.g. nanoemulsion) to encapsulate a dispersion of lipophilic bioactive compounds and optionally carrier solvent (hydrophobic inner core) in a hydrophilic outer shell material. The hydrophilic outer shell material is, optionally, food grade, does not adversely affect product quality (such as appearance, taste, texture, or stability), protected from chemical degradation during storage and distribution, and increases bioavailability following ingestion. Hydrophilic outer shell materials form a shell to help stabilize emulsions from Ostwald ripening, a destabilization mechanism of emulsions. This problem arises due to the increased solubility of dispersed phase in a hydrophilic solution.

The term “emulsion” is well known in the art and refers to a mixture of two or more liquids that are normally immiscible (unmixable or unblendable), where a first liquid is dispersed in small globules (internal or discontinuous phase) throughout a second liquid (external or continuous phase). In a nanoemulsion, the globules of the internal phase are on the nanoscale. Nanoemulsions are typically obtained by shearing a mixture comprising two immiscible liquid phases (for example, oil and water), one or more surfactants and, optionally, one or more co-surfactants.

In the context of the present disclosure, the physical structure of the emulsions described herein comprises at least two primary components. The first component is the core material, which can broadly be defined as the lipophilic interior phase (hydrophobic inner core) of the composition. In the case of the present disclosure, the core material comprises the cannabinoid or cannabis-derived compound, and optionally carrier solvent and other additives such as described herein. The second component is the wall material, which can broadly be defined as the hydrophilic outer shell of the composition comprising inulin and pectin. The physical structure of compositions comprising a core material encapsulated by an outer shell may be referred to as having a “core-shell structure”. When combined in appropriate proportions with the core material in a hydrophilic solvent (matrix) such as water, the wall material provides a surface for adsorption or, preferentially, acts as an external coating that encapsulates the core material.

The hydrophilic outer shell of the compositions of the present disclosure advantageously comprise inulin and pectin. In certain embodiments, other suitable hydrophilic outer shell materials may also be included, such as for example gum arabic, xanthan gum, locust bean gum, guar gum, gum tragacanth, gum karaya, tara gum, brea gum, gellan gum, mesquite gum, kappa carrageenan, lambda carrageenan, starch, octenyl succinic anhydride (OSA) starch, waxy maize starch, dextran, dextrin, cyclodextrin, maltodextrin, polydextrose, cellulose, microcrystalline cellulose, carboxymethyl cellulose, hydroxypropyl cellulose, hydroxypropyl methylcellulose, methyl cellulose, ethyl cellulose, inulin, fructooligosaccharide, alginic acid, sodium alginate, sugar beet pectin, citrus pectin, whey protein concentrate, whey protein isolate, casein, calcium caseinate, sodium caseinate, soy protein isolate, pea protein, hemp protein, rice-bran protein, egg albumin, gelatin, zein, corn protein, sucrose, glucose, fructose, lactose, galactose, maltose, chitin, chitosan, chitosan salts (e.g. hydrochloride, lactate, glutamate, and carboxymethyl), poly(lactic-co-glycolic acid) [PLGA], carnauba wax, agar, agarose, Konjac, sodium croscarmellose, glucomannan, polysorbate 80, sodium dodecyl sulfate, soy lecithin, sunflower lecithin, glycerol monostearate, polyvinyl pyrrolidone (PVP), hyaluronic acid, chondroitin sulfate, polyglutamic acid, amylose, amylopectin, soluble soybean polysaccharide, and phosphatidylcholine and combinations thereof.

The present disclosure is directed to compositions where the hydrophilic outer shell material includes inulin and pectin. In one embodiment, the hydrophilic outer shell material includes a combination of inulin and pectin in a weight ratio of inulin:pectin of from about 99%:1% w/w to about 60%:40% w/w. In some embodiments, the weight ratio of inulin:pectin is from about 99%:1% w/w to about 80%:20% w/w. In some embodiments, the weight ratio of inulin:pectin is between about 95%:5% w/w to about 60%:40% w/w, or between about 90%:10% w/w to about 70%:30% w/w, such as about 90%:10% w/w, about 85%:15% w/w, about 80%:20% w/w, about 75%:25% w/w, or about 70%:30% w/w. In some embodiments, the weight ratio of inulin:pectin is about 70%:30% w/w.

In an embodiment, the weight ratio of the hydrophilic outer shell material to the hydrophobic inner core material comprising the cannabinoid or cannabis-derived compound is between about 90%:10% w/w to about 50%:50% w/w. In some embodiments, the ratio of hydrophilic outer shell:hydrophobic inner core is between about 90%:10% w/w to about 70%:30% w/w. In some embodiments, the ratio of hydrophilic outer shell:hydrophobic inner core is between about 85%:15% w/w to about 70%:30% w/w. In select embodiments, such as where the cannabinoid comprises THC, the ratio of hydrophilic outer shell:hydrophobic inner core may preferably be between about 85%:15% w/w to about 75%:25% w/w, for example, about 80%:20% w/w. In other select embodiments, such as where the cannabinoid comprises CBD, the ratio of hydrophilic outer shell:hydrophobic inner core may be between about 80%:20% w/w to about 70%:30% w/w, for example, about 75%:25% w/w.

In one embodiment of the present disclosure, the composition comprises a weight ratio of inulin:pectin of between about 65%:35% w/w to about 75%:25%, a ratio of hydrophilic outer shell:hydrophobic inner core of between about 70%:30% w/w and about 85:15% w/w, and a weight ratio of carrier solvent:cannabinoid of between about 2:1 and 1:2. For example, in particular where the cannabinoid comprises THC, the composition may comprise a weight ratio of inulin:pectin of about 70%:30% w/w, a ratio of hydrophilic outer shell:hydrophobic inner core of about 80%:20% w/w, and a weight ratio of carrier solvent:cannabinoid of about 1:1 and 1:1. Alternatively, and in particular where the cannabinoid comprises CBD, the composition may comprise a weight ratio of inulin:pectin of about 70%:30% w/w, a ratio of hydrophilic outer shell:hydrophobic inner core of about 75%:25% w/w, and a weight ratio of carrier solvent:cannabinoid of about 1:1 and 1:1.

Encapsulation efficiency (EE) may be defined as the concentration of core material (cannabinoid, carrier solvent and/or other additives) in the composition over the initial concentration used to make the composition, and may be expression as a percentage. In essence, EE is a measure of how well a wall material can protect a core material from being dissolved in a (organic) solvent. A high EE indicates that the core material is fully encapsulated and is not dissolved or destabilized by solvent effects. A higher EE also improves the masking of cannabis taste and improves bioavailability of the cannabinoid or cannabis-derived compound. A high EE loading increases production throughput.

In embodiments of the present disclosure, the liquid formulations as described herein may be dried to form a powder formulation. As shown herein, the compositions of the present disclosure are capable of achieving high EE in the powder formulation (see Example 6). In an embodiment, the EE of the present compositions is at least 70%. In an embodiment, the EE of the present compositions is at least 85%. In an embodiment, the EE of the present compositions is at least 90%. In an embodiment, the EE of the present compositions is at least 95%. In an embodiment, the EE is at least 95% and the composition comprises an inulin:pectin ratio of about 70%:30% w/w and a ratio of hydrophilic outer shell:hydrophobic inner core of about 80%:20% w/w.

In some embodiments, the pre-bulked powders comprise at least 10% by weight of a cannabinoid or cannabis-derived compound, and including at least 15% by weight of a cannabinoid or cannabis-derived compound. In some embodiments, the powders include from 5% by weight to about 25% by weight cannabinoid or cannabis-derived compound, including from 5% by weight to about 20% by weight, and including from about 5% to about 15% by weight cannabinoid or cannabis-derived compound. In these embodiments, the cannabinoid(s) are typically THC and/or CBD.

Hydrophilic outer shells can provide an effect on the physicochemical stability of emulsions, nanoemulsions and/or microemulsions in the gastrointestinal tract (GI Tract). The rate and extent of lipid digestion is higher for medium chain triglyceride (MCT) emulsions than for long chain triglyceride (LCT) emulsions, which is attributed to differences in the water dispensability of the medium and long chain fatty acids formed during lipolysis. The total bioavailability of active components after digestion can be higher for LCT emulsions than for MCT emulsions.

Long-chain triglycerides (LCT) contain fatty acids of 12-20 carbon atoms, and can form mixed micelles with a hydrophobic/lipophilic inner core large enough to accommodate active substances such as THC and other cannabinoids, terpenoids and flavonoids. Medium-chain triglycerides (MCT) contain fatty acids of 12-20 carbon atoms and can form mixed micelles with smaller hydrophobic cores.

Emulsions can be prepared in concentrated form and later diluted several hundred times in sugar/acid solutions prior to consumption to produce finished products (e.g. beverages) in either carbonated or non-carbonated biocompatible matrix systems. Exemplary acids include, for example, citric acid, lactic acid, malic acid, ascorbic acid, sorbic acid, and the like and combinations thereof.

Selection of an emulsifier may affect the shelf-life and physicochemical properties of the emulsion. Emulsions stabilized by surfactants or other types of stabilizing agents phospholipids, amphiphilic proteins, or polysaccharides, have been developed to provide controlled release, improved entrapment efficiency, and protection from degradation.

Other suitable types of modifiers and additives include viscosity modifiers, natural emulsifiers, oils, thickening agents, minerals, acids, bases, vitamins, flavors, colourants, and the like and combinations thereof, as known in the beverage and food arts, to provide improved solubility, stability, bioavailability, colour and taste.

Emulsions can be prepared several ways such as mechanical processes which employs shear force to break large emulsion droplets into smaller ones, high-pressure homogenization (HPH, including microfluidization) and high-amplitude ultrasonic processing, and ultrasound-assisted emulsification. Suitably, the droplets are prepared to be in the nanometer size range, such as 100 nm or less.

Small droplet sizes lead to transparent emulsions. Droplet sizes about 100, 90, 80, 70, 60, 50 or 40 nm are desirable. Suitably the droplet sizes for transparent emulsions are in the range of 40 to 60 nm, more suitably they are 45 to 55 nm, more suitably yet, 50 nm.

Dosage Forms

A dosage form is that object delivered to a subject human or non-human organism for testing, placebo, recreational, therapeutic or other use. In an embodiment, the compositions of the present disclosure may be formulated as dosage forms for administration to a subject (e.g. the liquid or powder formulation within a soft gel capsule; a tablet comprising the powder formulation; the liquid or powder formulation absorbed onto or into a solid material).

Suitable dosages of the formulations will depend upon many factors including, for example, age and weight of an individual, at least one precise event requiring professional consultation, severity of an event, specific formulation to be used, nature of a formulation, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Suitable amounts of the cannabinoids or cannabis-derived compounds for use in the compositions of the present disclosure will depend upon many factors including, for example, age and weight of an individual, specific active compound(s) and/or additive(s) to be used, nature of a formulation, whether the composition is intended for direct administration or is a concentrate, and combinations thereof. Ultimately, a suitable amount can be readily determined by one skilled in the art. For example, one skilled in the art can begin with a low amount that can be increased until reaching the desired result or effect. Alternatively, one skilled in the art can begin with a high dosage that can be decreased until reaching a minimum dosage needed to achieve the desired result or effect.

In some embodiments, the dried powder formulation of the present disclosure can be formulated into pharmaceutical or recreational dosage forms comprising an effective amount of particles. Although mainly pharmaceutical dosage forms for oral administration such as tablets and capsules are envisaged, the particles of the present disclosure can also be used to prepare dosage forms e.g., for rectal administration. The compositions of the disclosure can also be used as a component of a dry powder inhaler or a solid liquid suspension in oil. Preferred dosage forms are those adapted for oral administration shaped as a tablet. They can be produced by conventional tableting techniques with conventional ingredients or excipients and with conventional tableting machines.

Tablet blends (including the powder formulations disclosed herein and any other conventional tablet ingredient or excipient) may be dry-granulated or wet-granulated before tableting. The tableting process itself is otherwise standard and readily practiced by molding a tablet from a desired blend or mixture of ingredients into the appropriate shape using a conventional tablet press.

Tablets may further be film-coated to improve taste or provide ease of swallowing and an elegant appearance. Many suitable polymeric film-coating materials are known in the art. A preferred film-coating material is hydroxypropyl methylcellulose HPMC, especially HPMC 2910 5 mPas. Other suitable film-forming polymers also may be used herein, including hydroxypropylcellulose and acrylate-methacrylate copolymers. Besides a film-forming polymer, the film coat may further comprise a plasticizer (e.g. propylene glycol) and, optionally, a pigment (e.g. titanium dioxide). The film-coating suspension also may contain talc as an anti-adhesive.

Foodstuffs and Beverages

The compositions of the present disclosure may be used in the preparation of foodstuffs and beverages. As used herein, a beverage is any drink that may be consumed by a subject. A foodstuff is any substance suitable for consumption as a food.

The compositions may be combined with any beverage-compatible or food-compatible ingredient. For example, liquid formulations of the present disclosure may be used directly in the preparation of foodstuffs and beverages, e.g. as an additive or ingredient. Powder compositions may be used either directly, e.g. as an additive or ingredient, or indirectly e.g. by first dissolving the powder in a solvent (e.g. water) to form a liquid composition prior to use. In some embodiments, the powder compositions may be added to beverage or foodstuff directly. In other embodiments, the powder compositions are diluted with a bulking agent. The pre-bulked and/or bulked powder compositions can be packaged for individual servings (e.g. sachets/packets), packages in bulk within a single container, or a combination thereof.

When used in beverages, the compositions of the present disclosure further comprise a beverage liquid. Generally, beverage liquids are liquids meeting the common meaning of the term “biocompatible”, which include materials that are not harmful to living tissue. Suitably, such beverage liquids comprise water, oil, alcohol; with or without additives or modifiers or both. Such beverage liquids can be divided into various groups such as plain water, alcohol, non-alcoholic drink, soft drink, fruit juice, vegetable juice, tea, coffee, milk, or other hot, room temperature or cold liquids used in drinks. Beverages can be caffeinated or non-caffeinated and may contain calories or not. Such beverages may be produced in ready to use form or be produced in a form suitable for preparation in final consumable form at or proximate to the time of ingestion.

Typically, beverage liquids will comprise about 50 weight or volume percent of the beverage, more typically, 50, 60, 70, 80, 90, 95 or 98% by weight or volume of the beverage. In one particular embodiment of the disclosure, the beverage liquid is about 95 to 98 weight or volume percent of the beverage.

Non-limiting examples of beverages that may be prepared with the compositions of the present disclosure include but are not limited to: hot and cold beverages including water, fruit juice, vegetable juice, tea, coffee, softs drinks, energy drinks, alcohol, flavoured water, or single-serve beverage cartridges. Non-limiting examples of foodstuffs include baked goods (e.g. cookies, brownies, cake, pie, biscuits and pastries), candies (e.g. hard candy, soft candy, gummies, etc.), chocolates, lozenges, gum, mints, dried fruits, nuts, granola, truffles, caramels, chews, taffy, prepared meals, cooking ingredients (e.g. food additives, dry spices, honey, sugar, sweeteners, etc.), ground coffee, instant coffee and tea leaves.

In an embodiment, the powder formulation of the present disclosure may be used in the preparation of a hard candy (e.g. similar to a Tic Tac™). The hard candy may include, for example, the powder formulation as disclosed herein, icing sugar, flavouring agents, magnesium stearate, an acidic component (e.g. citric acid) and/or a basic component (e.g. sodium bicarbonate), and any other suitable or appropriate additives. The ingredients may be mixed and then pressed into a candy shape to produce the hard candy. The hard candy may be of any suitable shape. In an embodiment, the powder formulation and the resulting hard candy may comprise THC. The powder formulations of the present disclosure advantageously allow for the preparation of hard candies with very specific concentrations of cannabinoids.

The amount of the composition of the present disclosure added to beverages or foodstuffs will vary depending on the desired dosage of cannabinoids (e.g. THC and CBD) or cannabis-derived compound. For example, in some embodiments each serving, unit or item of foodstuff or beverage will contain about 0.5 mg to about 100 mg of cannabinoids. In an embodiment, the foodstuff or beverage will contain about 2.0 mg to about 10 mg of cannabinoids. In an embodiment, the foodstuff or beverage will contain about 0.5 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, or about 10.0 mg of cannabinoids. In an embodiment, the cannabinoid is THC. In an embodiment, the cannabinoid is CBD.

Beverages may be packaged as individual packages, suitably single use packages, and multiple packages. The packaging can be in air tight containers. Packaging may be comprised of paper, plastic, metal, and glass. Beverages may include bubble containing or producing liquids with dissolved gas or liquids capable of producing gas proximately in time of consumption. In one embodiment of the disclosure, the beverages, optionally comprising additives, modifiers or both, are convenient to consumers and are manufactured at modest expense. Beverages with dissolved gas may be created by a method comprising addition of carbon dioxide, ozone, oxygen, and nitrogen. For beverages with dissolved gas, dissolved gas may be added to the beverage by methods comprising application of pressure, and adding water with the dissolved gas. The dissolved gas is released from the beverage when pressure is reduced as effervescence.

The compositions of the present disclosure are suitably low calorie, and can be used to prepare beverages and foodstuffs that are low calorie. Particularly, in some embodiments, a 250 mL or 2-5 g serving will provide less than 25 kilocalories (Kcal), more suitably less than 10 Kcal, and even more suitably less than 5 Kcal. Inulin is not digested in the upper gastrointestinal tract, and therefore has a low caloric value.

In some embodiments, the compositions, beverages and/or foodstuffs disclosed herein provide a desired intoxication effect as measured by a standard British unit of alcohol. As used herein, “one British unit of alcohol” is defined as 10 mL (8 g) of pure alcohol. That is the number of units of alcohol can be determined by multiplying the volume of the drink (in milliliters) by percentage ABV, and dividing by 1000.

Suitably, in some aspects, the beverages or foodstuffs are formed and administered to provide a subjective or objective intoxicating effect equivalent to a standard British unit of alcohol. More particularly, from about 25 mL to 500 mL of the beverage, more particularly, from about 35 ml to about 250 ml, and even more particularly, from about 60 ml to about 120 ml of the beverage, are formed and administered to provide an intoxicating effect equivalent to a standard British unit of alcohol. By further way of example, in one aspect, consuming about 35 mL to about 60 mL of the beverage causes either a subjective or objective intoxicating effect equivalent to a standard British unit of alcohol. In another aspect, consuming about 60 mL to about 120 mL of the beverage causes either a subjective or objective intoxicating effect equivalent to a standard British unit of alcohol. In yet another aspect, consuming about 120 mL to about 250 mL of the beverage causes either a subjective or objective intoxicating effect equivalent to a standard British unit of alcohol. In yet another aspect, consuming about 250 mL to about 500 mL of the beverage causes either a subjective or objective intoxicating effect equivalent to a standard British unit of alcohol.

It will further be appreciated that in certain embodiments the beverage or foodstuff should provide the human or non-human subject an intoxicating effect at the desired time. For example, in some embodiments, the beverage or foodstuff provides for an onset of intoxication in a time period of from about 10 minutes to about 120 minutes, including from about 20 minutes to about 90 minutes, and including from about 30 minutes to about 60 minutes, after consumption of the beverage or foodstuff. By way of further example, in certain embodiments the beverage or foodstuff can be formed and administered to provide for an onset of the intoxication of about 10 minutes, or about 15 minutes, or about 20 minutes, or about 25 minutes, or about 30 minutes, 40 minutes, 60 minutes, 90 minutes, or even 120 minutes. In further examples and embodiments, the beverage or foodstuff can be formed and administered to provide for an onset of the intoxication of about 180 minutes, or even about 240 minutes, or even still about 300 minutes.

Methods of Preparation

The compositions of the present disclosure may be prepared by combining a cannabinoid or a cannabis-derived compound with inulin and pectin, and homogenizing the mixture with a solvent to form an emulsion (e.g. a liquid formulation as described herein).

In a particular embodiment, the method comprises combining the inulin and pectin in a solvent (e.g. water) to form an inulin/pectin mixture (wall material); combining the cannabinoid or the cannabis-derived compound with the inulin/pectin mixture; and homogenizing the mixture to form an emulsion. In an embodiment, each of the combining steps are performed by mixing. In an embodiment, the cannabinoid or the cannabis-derived compound is mixed with a carrier solvent (e.g. oily medium) prior to combining with the inulin/pectin mixture. The cannabinoid alone or in admixture with the carrier solvent are embodiments of the core material.

The combination of the core material and wall material in a solvent such as water in appropriate proportion results in a stable emulsion after homogenization. “Homogenization” in the context of the present disclosure refers to the process of distributing the core material (e.g. cannabinoid, carrier solvent, and/or additives) in the solvent such that the wall material (comprising inulin and pectin) assembles to encapsulate the core material. In some embodiments, homogenizing comprises one or more of: magnetic stirring, high-shear mixing, microfluidizing, sonication, and ultrasonication. In some embodiments, the homogenization comprises a two-step process, wherein one of the steps is microfluidizing. For example, the emulsion may be homogenized by magnetic stirring and then microfluidizing.

In some embodiments, the emulsion is further dried (e.g. spray dried) to form a powder formulation. As described herein, the powder formulation may be used in the preparation of beverages and foodstuffs.

The liquid formulations may be dried using any method as known in the drying arts to evaporate the water phase of the emulsion, and possibly none, some or essentially all of the carrier solvent. For example, in one embodiment, the liquid formulations are spray dried to form the powder formulation. Alternative methods of preparing the dried powder formulation include, but are not limited to, pan coating, air-suspension coating, centrifugal extrusion, vibrational nozzle technique, freeze-drying or using a food dehydrator.

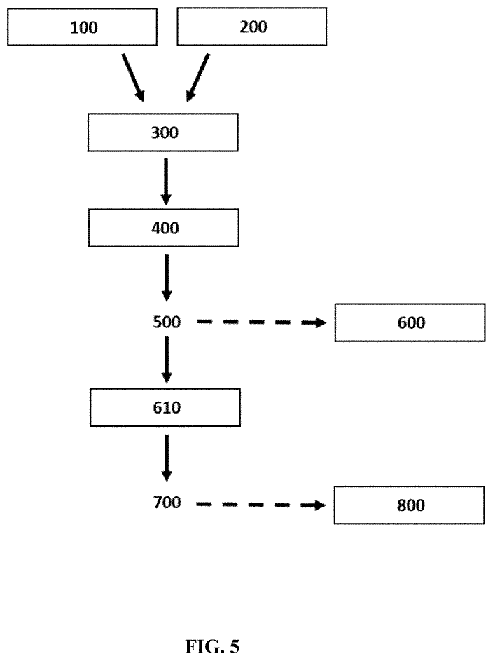

An embodiment of the methods of the present disclosure will now be described with reference to FIG. 5. The encapsulation material comprising inulin and pectin is mixed in water (100). The mixture may be stirred for up to 24 hours or until the material is dissolved. Alternatively, the mixture may be heated to assist dissolution (e.g. to about 60° C.). Separately, the cannabinoid, e.g. THC, and the carrier solvent, e.g. MCT oil, is combined and heated (200) until the cannabinoid is dissolved. The materials of steps (100) and (200) are then combined and emulsified (300).

In some embodiments, the wall material solution (inulin and pectin) from step 100 is poured into the core material solution (cannabinoid and carrier solvent) from step 200. This order of mixing in step 300 was found to reduce the cannabinoid adhering to the walls of the mixing container. In alternative embodiments, the core material solution (cannabinoid and carrier solvent) from step 200 is poured into the wall material solution (inulin and pectin) from step 100.

Emulsification in step 300 may be achieved using a homogenizer. The emulsion from step 300 is further homogenized using a microfluidizer (400) to reduce particle size and produce a liquid formulation 500 of the present disclosure, e.g. oil-in-water emulsion. The liquid formulation 500 may be incubated prior to use (600), such as in preparing a dosage form, beverage of foodstuff, or prior to drying (610).

The liquid formulation 500 may then be dried (610) to produce a powder formulation 700 using a spray dryer. The spray drying process 610 uses an atomizer or spray nozzle to disperse the liquid formulation into a controlled drop size spray or aerosol which is sprayed through a hot vapor stream (input gas) in a drying chamber. This causes the solvent in the emulsion to vaporize leaving the solid which collects in the collection cyclone. The powder formulation 700 may be used for preparing a dosage form, beverage or foodstuff as described herein (800).

In some embodiments, the concentration of inulin and pectin in the liquid formulation 500 prior to spray drying (610) is between about 4% w/w to about 15% w/w. For compositions comprising 30% pectin and above of encapsulation material (i.e. 70%:30% w/w inulin:pectin), a higher percentage of water in the liquid formulation 500 was found to improve the yield of the powder formulation 700. For example, the concentration of the inulin and pectin in the aqueous solvent may be between about 6% w/w to 10% w/w, between about 7% w/w to about 9% w/w, or about 7% w/w of inulin and pectin. This resulted in a stable, low viscosity liquid formulation 500 that had improved powder yield compared to comparative liquid formulations 500 comprising higher concentrations of inulin and pectin. However, an increase in yield must be balanced with avoiding a liquid formulation 500 that is too dilute, which could result in an unstable emulsion.

The inlet temperature of the spray dryer may be between about 100° C. and about 200° C. In some embodiments, the inlet temperature is between about 150° C. and about 200° C., or between about 150° C. and about 170° C. The input feed rate (QFlow) may be between about 10 and about 50, or between about 15 to about 45. The pump speed may be between about 10% (about 3 mL/min) to about 50% (about 15 mL/min), or between about 15% (about 5 mL/min) to about 20% (about 7 mL/min). In a particular embodiment, run parameters of the spray dryer may include an inlet temperature of about 150° C., an aspirator setting of 100%; a QFlow of about 35, and a pump speed of about 15% (about 5 mL/min).

In some embodiments, the liquid formulation 500 is incubated prior to spray drying (610). For example, the liquid formulation 500 may be incubated at room temperature for at least 24 hours, or up to 48 hours prior to spray drying (610).

In some embodiments, the powder formulation 700 obtained from drying (610) is milled to achieve a dry flowing powder. For example, the powder formulation 700 may be placed into a mill to granulize the solid. The mill may consist of a variety of apparatus including rotary blade mixing, conical mill, jet mill, hammer mill, mortar and pestle, auger type mixer as well as manual or automatic mixing, spreading or crushing of any kind. In some embodiments, the milled solid is further subjected to drying. For example, the milled solid may be placed in a vacuum oven to remove any remaining moisture.

In an alternative method of preparation, the core material and wall material are dissolved in an organic solvent such as ethanol within the same container. The resulting formulation can then be homogenized (300/400) and spray dried (610) to give a powder formulation 700.

Liquid formulations 500 of the present disclosure may be prepared from a mixture comprising cannabinoid or cannabis-derived compound, inulin and pectin in a solvent, e.g. water, which may be an emulsion. Liquid formulations of the present disclosure may also be prepared by dissolving the powder formulations of the present disclosure in a solvent, e.g. water, which may reform an emulsion.

Treatment Indications

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure. The following disorders are reported to be treated by use of cannabis or cannabinoid-containing formulations. There is no representation herein that cannabis or cannabinoid-containing formulations constituted a medically licensed treatment or cure for any of these disorders in any jurisdiction, only that published information exists for the use of cannabis or cannabinoid-containing formulations for the disorders recited below.

More particularly, in some embodiments, the compositions, dosage forms, beverages or foodstuffs of the present disclosure may be administered to a subset of individuals in need thereof as a therapeutic composition. As used herein, an “individual in need” refers to an individual at risk for or having a medical need such as those described herein. Additionally, an “individual in need” is also used herein to refer to an individual at risk for or diagnosed by a medical professional as having a condition described herein. As such, in some embodiments, the methods disclosed herein are directed to a subset of the general population such that, in these embodiments, not all the general population may benefit from the methods. Based on the foregoing, because some of the method embodiments of the present disclosure are directed to specific subsets or subclasses of identified individuals (that is, the subset or subclass of individuals “in need” of assistance in addressing one or more specific conditions noted herein), not all individuals will fall within the subset or subclass of individuals as described herein. Generally, the individual in need is a human. The individual in need can also be, for example, an animal such as a companion animal or a research animal such as, for example, a non-human primate, a mouse, a rat, a rabbit, a cow, a pig, and other types of research animals known to those skilled in the art.

These disorders include: ADD/ADHD, Addiction risk—Physical, Alcoholism, ALS, Alzheimer's, Amotivational Syndrome, Appetite Stimulant, Arthritis, Asthma, Atherosclerosis, Atrophie Blanche, Autism, Cancer—breast, Cancer—colorectal, Cancer—glioma/brain, Cancer—leukemia, Cancer—lung, Cancer—melanoma, Cancer—oral, Cancer—pancreatic, Cancer—prostate, Cancer—Skin, Cancer—Testicular, Chronic Cystitis, COPD, Diabetes, Depression, Dermatitis, Dystonia, Endocannabinoid Deficiency, Epilepsy, Familial Mediterranean Fever, Infertility, Fever, Fibromyalgia, Glaucoma, Heart Disease/Cardiovascular, Hepatitis, Herpes, Hiccups, HIV/AIDS, Hormone disorders, Huntington's Disease, Effects of Hysterectomy, Idiopathic Intracranial Hypertension, Meige's Syndrome, Migraine/Headache, multiple sclerosis, Nausea, Neuronal disorders, Neuropathic pain, Disorders treated by Neuroprotectants, Nutritional disorders, Obesity, Osteoporosis, Pain, Parkinson's Disease, Post-Traumatic Stress Disorder, Pregnancy related disorders, Pruritis, Schizophrenia/Mental disorders, Sickle Cell Disease, Sleep modulation, Spasticity, Spinal Cord Injury, Stroke, Tourette's Syndrome, and Wilson's Disease.

Particularly suitable embodiments of disorders treated by cannabis and cannabinoid-containing formulations include: Acne, ADD and ADHD, Addiction, AIDS, ALS, Alzheimer's Disease, Anorexia, Antibiotic Resistance, Anxiety, Atherosclerosis, Arthritis, Asthma, Autism, Bipolar disorder, Cancer, Digestive Issues, Depression, Diabetes, Endocrine Disorders, Epilepsy and Seizures, Fibromyalgia, Glaucoma, Heart Disease, Huntington's Disease, Inflammation, Irritable Bowel Syndrome, Kidney Disease, Liver Disease, Metabolic Syndrome, Migraine, Mood Disorders, Motion Sickness, Multiple Sclerosis (MS), Nausea, Neurodegeneration, Chronic Pain, Obesity, OCD, Osteoporosis/Bone Health, Parkinson's Disease, Prion/Mad Cow disease, PTSD, Rheumatism, Schizophrenia, Sickle Cell Anemia, Skin Conditions, Sleep Disorders, Spinal Cord Injury, Stress, Stroke and TBI.

### EXAMPLES

The following examples are included to demonstrate various embodiments of the present disclosure. It should be appreciated by those of skill in the art that the techniques disclosed in the examples that follow represent techniques discovered by the inventors to function well in the practice of the present disclosure, and thus may be considered to constitute preferred modes for its practice. However, those of skill in the art should, in light of the present disclosure, appreciate that many changes can be made in the specific embodiments which are disclosed and still obtain a like or similar result without departing from the scope of the present disclosure.

### Example 1

In this Example, a powder formulation including THC was prepared.

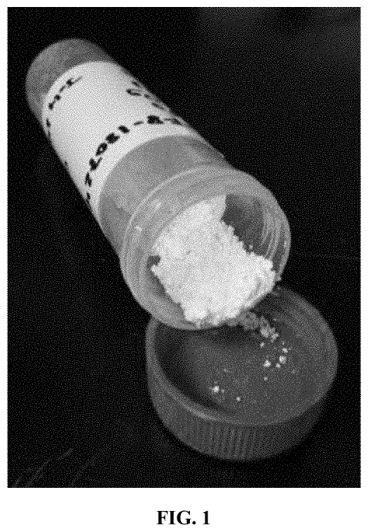

Initially, inulin (19 g) and pectin (1 g) (95%/5%) were dissolved in water (Milli-Q, 120 mL) and the mixture was stirred for 24 hours using a magnetic stir plate and stir bar. THC distillate (2 g) was added to organic coconut oil (2 g) in a beaker and the mixture was heated at 60° C. on a hot plate while manually stirring with a spatula until a homogenous oil formed. The inulin/pectin solution was added to the THC distillate/oil solution and the mixture was homogenized at 10,000 rpm for 5 minutes using a high-shear homogenizer (KIN EMATICA, Polytron PT 2500E). The mixture was further homogenized at a maximum pressure of 30,000 psi for 10 minutes using a microfluidizer (Microfluidics, M-110P). The resulting milky emulsion was spray dried using a Buchi B-290 Mini Spray Dryer with an input temperature of 150° C., an output temperature of 83° C., gas flow of 414 L/h and a feed rate of 5 mL/min with an aspirator setting of 100% to afford a white, dry free-flowing powder formulation (FIG. 1).

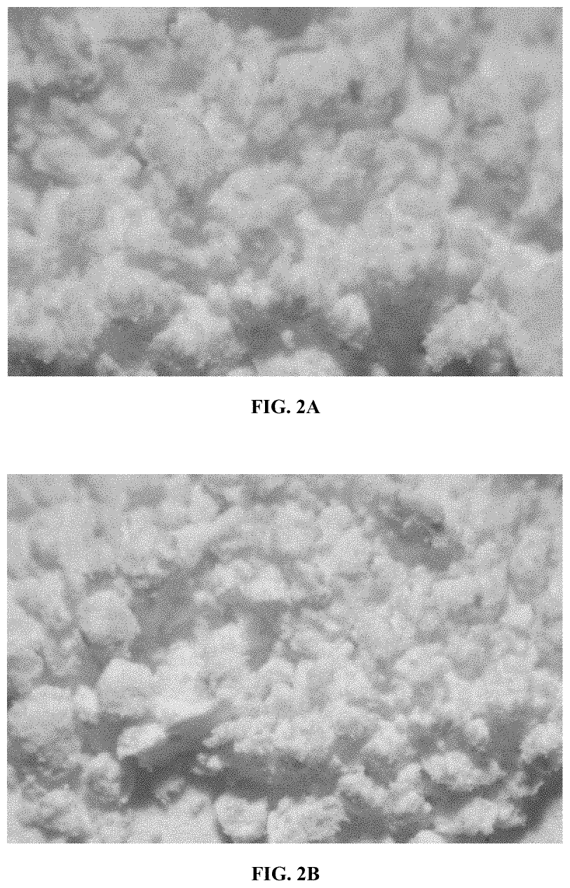

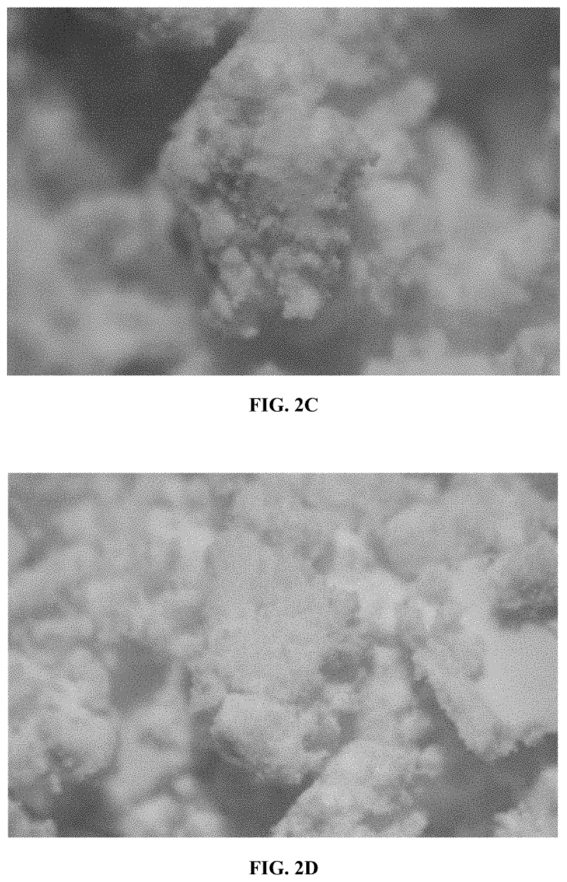

The powder was viewed directly under a stereoscopic microscope (Zeiss Stemi DV4) at 8× magnification (see FIGS. 2A and 2B) and 32× magnification (see FIGS. 2C and 2D).

### Example 2

In this Example, the powder formulations of Example 1 were analyzed for their abilities to dissolve in an aqueous solution.

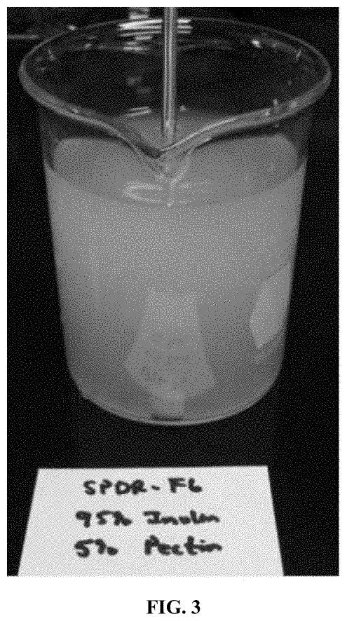

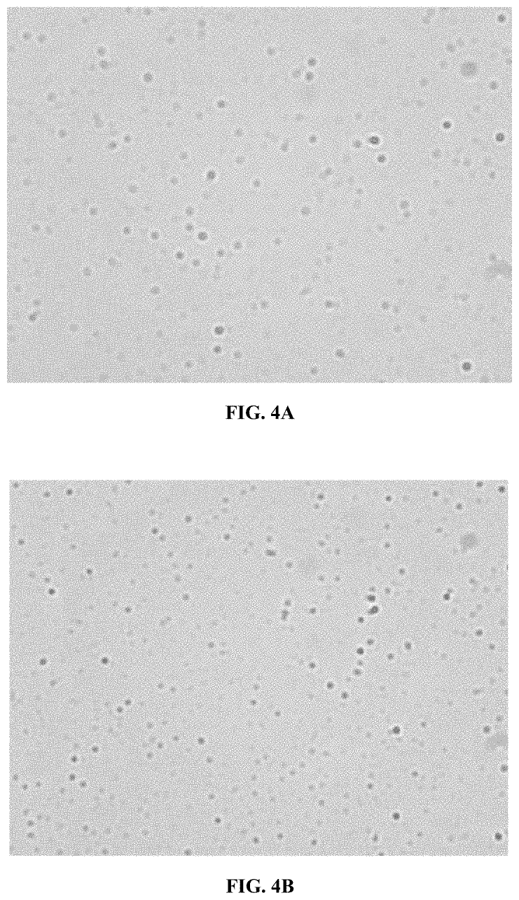

First, 250 mg of the powder obtained from the 95% Inulin/5% Pectin formulation was mixed with 4.75 g of erythritol and the mixture was added to 200 mL of water and stirred. This process resulted in full dissolution within 30 seconds (see FIG. 3). The powder was placed on a compound microscope (Zeiss, Axio Lab.A1, 10× ocular lens and 100× objective lens=1000× magnification) and analyzed. The results are shown in FIGS. 4A and 4B.

Additionally, 250 mg of powder obtained from a 35% Gum Arabic/65% Inulin formulation was added to 200 mL of water and stirred. This process resulted in full dissolution within 60 seconds.

### Example 3

In this Example, powder formulations were prepared using different microencapsulation materials and analyzed for their abilities to dissolve in an aqueous solution.

Emulsions were prepared by adding 20 grams of microencapsulation material to 120 mL of water. The microencapsulation mixture was stirred at room temperature for 24 hours using a magnetic stir plate and stir bar. THC distillate (2 grams) and organic coconut oil (2 grams) were combined in a glass beaker and heated at 60° C. for 15 minutes. The THC/oil mixture was manually stirred with a metal spatula to form a homogenous solution. The microencapsulation mixture was added to the THC/oil mixture and the resultant emulsion was homogenized at 10,000 rpm for 5 minutes using a benchtop homogenizer. The emulsion was then transferred to a high pressure microfluidizer and further homogenized to reduce sample viscosity. To form powders, the emulsion was spray dried using a Buchi B-290 Mini Spray Dryer (Temperature in (Tin)=150° C.; Temperature out (Tout)=83° C.); Aspirator=100%; Spray gas flow=414 L/h (35 mm); Feed rate (QFlow)=15.

Seven formulations were prepared: (1) 100% gum arabic (control); (2) 50% gum arabic/50% inulin; (3) 34% gum arabic/66% inulin; (4) 95% inulin/5% pectin; (5) 90% inulin/10% pectin; (6) 63% inulin/32% gum arabic/5% pectin; (7) 95% inulin/5% sodium alginate. All of the seven formulations resulted in the formation of white powders that had similar appearance and powder flow.

To analyze water solubility/dispersibility, 250 mg of powder was added to 200 mL of water and stirred for 1 minute. The formulations including 95% inulin/5% pectin (formulation (4)) and 34% gum arabic/66% inulin (formulation (3)) fully dispersed within 1 minute.

To improve solubility/dispersibility, a bulking agent was added to the powder. Specifically, 250 mg of spray dried powder was mixed with 4.75 grams of sucrose and added to 200 mL of water. The formulation including 95% inulin/5% pectin (formulation (4)) fully dispersed after 20 seconds of stirring.

### Example 4

In this Example, a process for preparing a composition according to the present disclosure is described, whereby 70%:30% w/w inulin:pectin were used as microencapsulation materials to prepare a composition comprising 80%:20% w/w pectin+inulin:THCd+MCTo.

Emulsion Preparation

An emulsion was prepared by adding inulin (14 g) and pectin (6 g) (30% pectin) to 120 mL of water (pH₂O), to give a concentration of pectin+inulin of 14.3% w/w (4.3% w/w pectin). The mixture was stirred at room temperature for 24 hours using a magnetic stir plate and stir bar.

THC distillate (2.5 g; THCd) and MCT oil (2.5 g; MCTo) were combined in a glass beaker and heated at 60° C. for 15 minutes. The THCd/MCTo mixture was manually stirred with a metal spatula to form a homogenous solution.

The THCd/MCTo mixture was added to the inulin/pectin mixture and emulsified using a benchtop homogenizer. Emulsification produced a white-beige emulsion, moderately viscous, totaling about 140 mL. The emulsion was then microfluidized to produce a milky white emulsion, of slightly lower viscosity, having a total volume of about 85 mL.

Powder Preparation

The microemulsion was spray dried using a Buchi B-290 Mini Spray Dryer (Temperature in (Tin)=150° C.; Temperature out (Tout)=83° C.); Aspirator=100%; Spray gas flow=414 L/h (35 mm); Feed rate (QFlow)=15.

Spray drying the microemulsion produced 6.30 g of white powder with a slight hint of beige. The powder was dry and granular. The powder had a normal distribution/coating in the primary cyclone of the spray dryer with minimal build up on the collection cyclone or its inlets. The majority of the powder was found in the collection flask. Powder was retrieved, stored and tested.

The process described in this example for a composition comprising 70%:30% w/w inulin:pectin and 80%:20% w/w pectin+inulin:THCd+MCTo, was adapted by varying the ratios of various components to produce alternative compositions of the present disclosure (see Table 9). The process parameters were the same unless otherwise stated.

For compositions comprising less than 30% pectin, it was preferred to increase QFlow to 20 and increase pump speed to 40% (about 12.5 mL/min). Alternatively, doubling the water content of the inulin+pectin solution was found to provide improved powder yield for a QFlow of 35 and pump speed of 20% (about 7 mL/min) (see Example 9).

### Example 5

In this Example, liquid formulations (emulsions) were prepared according to the method described in Example 4, except that the ratios of inulin:pectin and ratios of core material:wall material were varied (see Table 2). The stability of the emulsions was observed while varying composition parameters to identify potential upper limits of the emulsion capacity of pectin. The results show that around 37.5% core loading appears to be about the upper limit for emulsion capacity for a wall comprising 95:5 inulin:pectin (e.g. to produce a stable emulsion). Similarly, around 50% core loading appears to be about the upper limit for emulsion capacity for a wall comprising 90:10 inulin:pectin, and around 70% core loading for a wall comprising 85:15 inulin:pectin. For compositions comprising 70:30 inulin:pectin, the core loading upper limit for emulsion capacity is above 43%.

TABLE 2

| Wall | Core | Total | | |
|---|---|---|---|---|
| composition | composition | material | | |
| (Inulin:pectin) | (THC:MCT oil) | (g) | Wall:core | Visual Inspection |
| 95:5 | 1:1 | 8 | 3:1 | Small number of oil droplets; still |
| | | | (25% core) | suitable for spray drying; near upper |
| | | | | limit for emulsion capacity. |
| 95:5 | 1:1 | 9.6 | 6:3.6 | Significant number of oil droplets; |
| | | | (37.5% core) | above upper limit for emulsion |
| | | | | capacity. |
| 90:10 | 1:1 | 9.6 | 6:3.6 | Stable after 1 hour; well below upper |
| | | | (37.5% core) | limit for emulsion capacity. |
| 90:10 | 1:1 | 12 | 6:6 | Small number oil droplets; still |
| | | | (50% core) | suitable for spray drying; near upper |
| | | | | limit for emulsion capacity. |
| 85:15 | 1:1 | 15.1 | 6:9.1 | Small droplets, no indication of |
| | | | (60% core) | phase separation; well below upper |
| | | | | limit for emulsion capacity. |
| 85:15 | 1:1 | 20.2 | 6:14.2 | Small droplets, no phase separation; |
| | | | (70% core) | near upper limit for emulsion |
| | | | | capacity. |
| 90:10 | 2:1 | 13.2 | 1:1 | Small oil droplets visible; phase |
| | | | | separation after 90 minutes; above |
| | | | | emulsion capacity. |
| 85:15 | 2:1 | 12.32 | 1:1 | Small oil droplets visible; no phase |
| | | | | separation; below upper limit for |
| | | | | emulsion capacity. |
| 90:10 | 1:1 | 25.8 | 1:1 | 100 mM CaCl₂ was added to the |
| | | | | emulsion. Some oil droplets were |
| | | | | observed; no phase separation. |
| 70:30 | Pure THC | 12.86 | 9:3.86 | No oil droplets or phase separation. |
| | | | (43% core) | |

### Example 6

In this Example, powder formulations were prepared according to the method described in Example 4, except that the ratios of inulin:pectin and ratios of core material:wall material were varied (see Table 3). The core materials comprised a 1:1 ratio of THC distillate:MCT oil unless otherwise stated. (P) indicates formulations using 100% API Active Materials (pure THC). (GA) refers to guar arabic.

TABLE 3

| Wall | Core | Total | | |
|---|---|---|---|---|
| composition | composition | material | | |
| (Inulin:pectin) | (THC:MCT oil) | (g) | Wall:core | Visual Inspection |
| 100% GA | 1:1 | 28.57 | 20:8.57 | White, slightly beige powder, not quite |
| | | | | “dry”; normal distribution on primary |
| | | | | cyclone, collection cyclone almost |
| | | | | entirely clear of powder. |
| 90:10 | 1:1 | 25 | 20:5 | White, slightly clumpy powder; normal |
| | | | | distribution in primary and collection |
| | | | | cyclones and inlets. |
| 80:20 | 1:1 | 25 | 20:5 | White, slightly beige clumpy powder. |
| 70:30 | 1:1 | 25 | 20:5 | White, slightly beige clumpy powder. |
| 70:30 | 1:1 | 26.67 | 20:6.67 | White, slightly beige powder, dry and |
| | | | | granular; normal distribution in primary |
| | | | | cyclone, collection cyclone almost |
| | | | | entirely clear. |
| 70:30 | 1:1 | 28.57 | 20:8.57 | White, slightly beige powder, not quite |
| | | | | “dry”; normal distribution in primary |
| | | | | cyclone, collection cyclone almost |
| | | | | entirely clear of powder. |
| 70:30 | 1:1 | 33.3 | 20:13.3 | Off-white powder, dry and static; |
| | | | | normal distribution on cyclone spray |
| | | | | drying. |
| 70:30 | Pure THC (P) | 23.53 | 20:3.53 | White slightly beige/yellow powder; |
| | | | | normal distribution in primary cyclone, |
| | | | | significant yellow build up near top, |
| | | | | most powder found in collection flask. |
| 70:30 | Pure THC (P) | 25 | 20:5 | Normal distribution on primary cyclone, |
| | | | | significant yellow build up at top, most |
| | | | | powder found in collection flask. |

Encapsulation efficiency (EE %) and cannabinoid loading (API %) of the powders were assessed and the results are shown in Table 4 below. The results show that at 20% core loading, increasing the percentage of pectin in the wall materials increases encapsulation efficiency. For formulations comprising 30% pectin as wall material (i.e. 70%:30% w/w inulin:pectin), increasing the core loading beyond 20% was found to decrease encapsulation efficiency. Removing MCT oil from the core materials resulted in an increased API %, but led to a decrease in encapsulation efficiency.

Encapsulation Efficiency Testing

The spray dried powders were washed with pentane via vacuum filtration and the resulting extracted oils (e.g. THC distillate and MCT oil) were weighed.

Encapsulation efficiency was calculated by comparing the mass of the input active materials (THC distillate and MCT oil) to the mass of the extracted oils (see Equation 1).

$$Encapsulation Efficiency ( \% ) = \frac{Extract mass ( g )}{Initial powder mass ( g ) \times ( \frac{Active weight ( \% )}{100} )} \times 100 \qquad Equation 1$$

TABLE 4

| Wall | Core | Encapsulation | API % |
|---|---|---|---|
| materials | loading | Efficiency | (input |
| (% pectin) | (%) | (%) | API × EE %) |
| 100% GA | 30 | 82.1 | 9.9 |
| 10 | 20 | 67.2 | 5.4 |
| 20 | 20 | 70.6 | 5.6 |
| 30 | 20 | 96.2 | 7.7 |
| 30 | 25 | 85.2 | 8.5 |
| 30 | 30 | 73.2 | 8.8 |
| 30 | 40 | 52.5 | 8.4 |
| 30 | 15 (P) | 88.1 | 10.6 |
| 30 | 20 (P) | 67.1 | 10.7 |

The results in Table 4 show that the compositions of the present disclosure were capable of achieving high encapsulation efficiencies. In particular, with about 20% to 30% pectin in the wall material and a core loading of between about 20% and about 30%, the EE was consistently greater than 70%. Notably, with about 30% pectin in the wall material and a core loading of between about 20% and about 25%, the EE was greater than 85%.

The results in Table 4 further show that the “pure” (P) API core loading formulations (i.e. no carrier solvent in the core materials), even though they can have a lower EE (e.g. bottom row at 67.1% EE), can still have a high effective API % due to how concentrated the API is the core material.

The API % in Table 4 is effectively a measure of the actual amount of API encapsulated in the powder. For example, taking the first row, core loading is 30% at 1:1 THC distillate (API) to carrier oil, and the THC distillate contained about 80% w/w THC. Therefore, the actual API % is the core loading percent (30), multiplied by the percent API in the core material (1:1=50%), multiplied by concentration of the API (80% w/w THC), multiplied by the encapsulation efficiency (82.1%) (30×0.5×0.8×0.821=9.9).

### Example 7

In this Example, powder formulations were prepared according to the methods described in Example 4, except that the THCd in the core was replaced with cannabidiol isolate (CBDi) or limonene (a terpene) as shown in Table 5.

The only notable difference in the preparation method was that the water content of the emulsion prepared prior to spray drying was doubled, such that the concentration of wall materials (inulin+pectin) in solution was around 7.6%.

For compositions comprising limonene, the spray dryer inlet temperature was set to 130° C. and the pump rate was set to 10% (about 5 mL/min). CBDi was found to readily mix with MCTo with the application of mild heat. This is in contrast to THCd which required longer heating and stirring to ensure mixing.

TABLE 5

| Wall | | Total | | |
|---|---|---|---|---|
| composition | Core | material | | |
| (Inulin:pectin) | composition | (g) | Wall:core | Visual inspection notes |
| 70:30 | 1:1 | 25 | 20:5 | White, slightly beige powder, dry not |
| | (CBDi:MCTo) | | (20% core) | granular; normal distribution, most in |
| | | | | collection flask. |
| 70:30 | 1:1 | 26.7 | 20:6.7 | White, slightly beige powder, dry not |
| | (CBDi:MCTo) | | (25% core) | granular; normal distribution, most in |
| | | | | collection flask. |
| 70:30 | 1:1 | 28.57 | 20:8.57 | White, slightly beige powder, dry not |
| | (CBDi:MCTo) | | (30% core) | granular; normal distribution, most in |
| | | | | collection flask. |
| 70:30 | Pure R-(+)- | 25 | 20:5 | White, clumpy powder smelling of |
| | Limonene | | | lemons; normal distribution, inlets and |
| | | | | collection cyclone clear until H₂O purge, |
| | | | | then most powder in collection flask. |
| 70:30 | Pure R-(+)- | 21.1 | 20:1.1 | White, dry, granular powder, smelling |
| | Limonene | | | slightly of lemons; normal distribution, |
| | | | | inlets and collection cyclone clear until |
| | | | | H₂O purge, then most powder in |
| | | | | collection flask. |

The encapsulation efficiency (EE %) of powders comprising CBD was assessed at different core loadings and the results are shown in Table 6. The peak encapsulation efficiency for CBD formulations was found to be around 25% core loading. This is in slight contrast to THC containing powders where a peak encapsulation efficiency of 96.2% was achieved at core loadings of 20% (see Table 6).

TABLE 6

| Core loading (%) | Encapsulation efficiency (%) |
|---|---|
| 20 | 86.1 |
| 25 | 89.1 |
| 30 | 82.4 |

### Example 8

In this Example, powder formulations were prepared according to the method described in Example 4, except that a bulking agent was added either during emulsion formation or after obtaining the spray dried powder. The effect of including the bulking agent with the spray dried powder on encapsulation efficiency was evaluated.

Addition of Myo-Inositol Prior to Encapsulation

The formulation was prepared using 7.82 g of wall material (inulin+pectin) and 1.96 g core material (0.98 g THCd and 0.98 g MCTo). The wall solution consisted of 70%:30% inulin:pectin in 260 mL of pH₂O, with a wall material (inulin+pectin) concentration of 14.3% w/w. 49 mL of this wall solution was used along with 101 mL additional pH₂O and 21.38 g of myo-inositol (bulking agent) giving 3.1% w/w of THCd in the solid material. The total pH₂O in the solution was 150 mL.

Spray drying the microemulsion produced 10.1 g of white powder. The powder had a normal distribution/coating in the primary cyclone and inlets with minimal build up on the collection cyclone. Powder in the collection flask was retrieved for testing.

Addition of Myo-Inositol to Spray Dried Powders

Powder formulations were prepared as described in Example 4. Around 150 mg of spray dried powder was required to achieve a 10 mg API dosage (the amount of powder to achieve a 10 mg API dosage may vary depending on the API %). The powders were made up to 1 g with myo-inositol using different mixing methods. The final powders were tested for dissolution properties and encapsulation efficiency (see Table 7). To analyze water solubility/dispersibility, 250 mg of powder was added to 200 mL of water and stirred for 1 minute. Encapsulation efficiency was calculated as described in Example 6.

TABLE 7

| | Dissolution | Oil observed at | Encapsulation |
|---|---|---|---|
| | in | solution | efficiency |
| Mixing method | water (s) | surface? | after mixing |
| Manual shaking in | 30-40 | No | 94.2% |
| closed container to | | | |
| mimic V-blender | | | |
| Mortar and pestle | <15 | Yes | 76.9% |
| Magic bullet blending | <15 | Yes | 83.9% |

Myo-inositol was found to incorporate well into the compositions prior to spray drying as well as after spray drying. When mixing myo-inositol with the spray dried powder compositions, it was found that the dissolution of spray dried powders in water could be improved if the mixing was carried out using a pestle and mortar method or magic bullet blending method. In addition to its properties as a natural, low-calorie sweetener, these results confirm that myo-inositol is a highly suitable bulking agent for oral compositions.

### Example 9

In this Example, powder formulations were prepared according to the method described in Example 4, except that certain preparation conditions were varied. All formulations comprised a wall composition of 70%:30% w/w inulin:pectin, 20% of core material (i.e. wall:core ratio 4:1) and a 1:1 ratio of THCd:MCTo.

The control emulsion is the emulsion prepared as described in Example 4 (also see Table 4) with an inulin+pectin solution having a concentration of 14.3% w/w inulin+pectin.

TABLE 8

| | | Spray | |
|---|---|---|---|
| | | Dried | |
| | Encapsulation | Powder | |
| Conditions | Efficiency % | Yield % | Observations |
| Control | 96.2 | 100 | |
| x2 water content | 93.5 | 148 | Low viscosity |
| | | | emulsion, increased |
| | | | yield. |
| x4 water content | 92.2 | 173 | Low viscosity |
| | | | emulsion, active |
| | | | materials crash out |
| | | | of emulsion, |
| | | | increased yield. |
| Spray dryer | 88.3 | 92.3 | Water droplets |
| temperature | | | visible in drying |
| reduced | | | chamber. |
| (inlet = 130° C.; | | | |
| outlet = 78° C.) | | | |
| Spray dryer | 91.0 | 87.3 | No significant change. |
| temperature | | | |
| increased | | | |
| (inlet = 170° C.; | | | |
| outlet = 78° C.) | | | |
| Increase input feed | 81.5 | 104 | No significant change. |
| rate (QFlow) to 45 | | | |
| Increase input feed | 95.1 | 78.8 | Spray drying |
| rate (QFlow) to | | | completed |
| 45 & heated | | | x5 faster |
| emulsion feed | | | with lower yield. |
| x2 water content & | — | 59.0 | Spray drying |
| increase input | | | completed x5 faster, |
| feed rate | | | water droplets |
| (QFlow) to 45 | | | visible in drying |
| | | | chamber & hard |
| | | | yellow build up in |
| | | | collection cyclone. |
| | | | Reduced yield. |
| x2 water content & | — | 58.0 | Spray drying |
| increase | | | completed x5 faster, |
| input feed rate | | | no water droplets |
| (QFlow) to 45 | | | or hard yellow |
| & inlet | | | build up. Reduced |
| temperature | | | yield. |
| increased to 200° C. | | | |
| x2 water content & | 87.4 | 97.7 | No significant change. |
| 12 hour RT dissolution | | | |
| of wall materials | | | |
| x2 water content & | 91.1 | 110 | No significant change. |
| 60° C. dissolution | | | |
| of wall materials | | | |
| x2 water content & | 99.0 | 109 | No change in |
| 44 hour RT | | | viscosity after |
| incubation of | | | incubation, increase |
| emulsion | | | in EE %. |

Varying Water Content

The results show that when the water content of the emulsion was doubled (×2), emulsion viscosity was lowered and powder yield was increased. However, diluting the emulsion further (×4) resulted in a less optimal emulsion which was observed by oils crashing out of the emulsion solution.

Spray Dryer Temperature

The results show that lowering the inlet temperature of the spray dryer to 130° C. resulted in water droplets visible in the drying chamber. Otherwise, no other significant changes to the spray drying process were observed.

Input Feed Rate (QFlow)

For formulations with a water content as described in Example 4, the results show that increasing the input feed rate of the spray dryer did not have a significant effect on the process. However, heating the emulsion while it was fed into the spray dryer resulted in faster spray drying (×5 faster) but with lower yields.

For formulations with double the water content (×2), the results show that increasing the input feed rate of the spray dryer completed the spray drying ×5 faster, however yield was decreased and a hard yellow build up accumulated in the collection cyclone. Increasing the spray dryer temperature eliminated the hard yellow build up and maintained the ×5 faster spray drying, but yield was still reduced.

Dissolution of Wall Materials

The results show that heating the solution of wall materials (inulin+pectin) in water or stirring the wall materials at room temperature overnight had no significant effect on encapsulation efficiency.

Incubation of Microfluidized Emulsion

The results show that room temperature incubation had no effect on emulsion viscosity. On the other hand, encapsulation efficiency was found to increase to 99%.

### Example 10

The following formulations described in Table 9 were prepared according to the method described in Example 4, except that certain parameters were varied as indicated. Spray drying had the following parameters unless otherwise states: QFlow=15; pump speed=20% (about 7 mL/min); inlet temperature=150° C.; aspirator 100%. The emulsions were all stable and produced off-white powders after spray drying. The resulting powder formulations were suitable for addition to foodstuffs and beverages.

TABLE 9

| Wall | Core | Total | | Powder | | | |
|---|---|---|---|---|---|---|---|
| (Inulin:pectin) | (THCd:MCTo) | material (g) | Wall:core | Yield (g) | | Notes | Variable |
| 90:10 | 1:1 | 25.09 | 17.56:7.53 | 3.5 | g | White clumpy | 30% active:wall |
| | | | | | | powder; not damp; | |
| | | | | | | normal distribution of | |
| | | | | | | powder spray drying | |
| 90:10 | 1:1 | 25.9 | 19.15:6.75 | 5.73 | g | White clumpy | 26% active:wall |
| | | | | | | powder; not damp; | |
| | | | | | | normal distribution of | |
| | | | | | | powder spray drying | |
| 90:10 | — | | 1:0 | 4.92 | g | White granular | Control |
| | | | | | | powder, spray dry | (0% active) |
| | | | | | | powder primarily in | |
| | | | | | | collection flask, no | |
| | | | | | | build up in cyclone | |
| 90:10 | 1:1 | 25.8 | 1:1 | 1.60 | g | Emulsion after | Addition of |
| | | | | | | microfluidization | calcium (CaCl₂) |
| | | | | | | unstable and began | |
| | | | | | | to gel so spray dried | |
| | | | | | | rapidly. | |
| | | | | | | White powder, not | |
| | | | | | | damp, normal | |
| | | | | | | distribution in spray | |
| | | | | | | dryer, some coating | |
| | | | | | | on cyclone and inlets. | |
| 60:40 | 1:1 | 40.0 | 1:1 | 10.6 | | Off-white damp | 40% pectin & |
| | | | | | | powder; significant | 50% active:wall |
| | | | | | | build up of powder in | |
| | | | | | | cyclone and inlets | |
| 90:10 | 1:1 | 33.3 | 20:13.3 | 8.66 | g | Yellow-white powder, | 10% pectin & |
| | | | | | | slightly damp; normal | 40% active:wall |
| | | | | | | distribution in spray | |
| | | | | | | drying, some build up | |
| | | | | | | on inlets | |
| 70:30 | 1:1 | 33.3 | 20:13.3 | 6.47 | g | Off-white powder, dry | 40% active:wall |
| | | | | | | and static; normal | |
| | | | | | | distribution on | |
| | | | | | | cyclone spray drying | |
| 70:30 | 1:1 | 28.57 | 20:8.57 | 5.51 | g | White, slightly beige | 30% active:wall |
| | | | | | | powder, not quite | |
| | | | | | | “dry”; normal | |
| | | | | | | distribution in primary | |
| | | | | | | cyclone, collection | |
| | | | | | | cyclone almost | |
| | | | | | | entirely clear of | |
| | | | | | | powder | |
| 100% GA | 1:1 | 28.57 | 20:8.57 | 9.12 | g | White, slightly beige | Control GA wall |
| | | | | | | powder, not quite | (30% active:wall) |
| | | | | | | “dry”; normal | |
| | | | | | | distribution on | |
| | | | | | | primary cyclone, | |
| | | | | | | collection cyclone | |
| | | | | | | almost entirely clear | |
| | | | | | | of powder. | |
| 70:30 | Pure THC | 12.86 | 9:3.86 | 1.07 | g | White, slightly beige | Pure THC |
| | | | | | | powder, dry and | |
| | | | | | | granular; most | |
| | | | | | | powder found in | |
| | | | | | | collection cyclone | |
| 70:30 | 1:1 | 25 | 20:5 | 6.07 | g | White, slightly beige | 20% active:wall |
| | | | | | | powder, dry and | |
| | | | | | | granular. | |
| 70:30 | 1:1 | 26.67 | 20:6.67 | 6.21 | | White, slightly beige | 25% active:wall |
| | | | | | | powder, dry and | |
| | | | | | | granular; normal | |
| | | | | | | distribution in primary | |
| | | | | | | cyclone, collection | |
| | | | | | | cyclone almost | |
| | | | | | | entirely clear. | |
| 70:30 | Pure THC | 25 | 20:5 | 7.28 | | Normal distribution on | 20% active:wall |
| | | | | | | primary cyclone, | & Pure THC |
| | | | | | | significant yellow | |
| | | | | | | build up at top, most | |
| | | | | | | powder found in | |
| | | | | | | collection flask | |
| 70:30 | Pure THC | 23.53 | 20:3.53 | 5.42 | | White slightly | 15% active:wall |
| | | | | | | beige/yellow powder; | & Pure THC |
| | | | | | | normal distribution in | |
| | | | | | | primary cyclone, | |
| | | | | | | significant yellow | |
| | | | | | | build up near top, | |
| | | | | | | most powder found in | |
| | | | | | | collection flask | |
| 90:10 | 1:1 | 25 | 20:5 | 8.94 | g | White, slightly clumpy | 10% pectin |
| | | | | | | powder; normal | |
| | | | | | | distribution in primary | |
| | | | | | | and collection | |
| | | | | | | cyclones and inlets. | |
| 80:20 | 1:1 | 25 | 20:5 | 6.45 | g | White, slightly beige | 20% pectin |
| | | | | | | clumpy powder. | |
| 70:30 | 1:1 | 33.4 | 16.7:16.7 | 4.62 | g | White powder, yellow | 30% pectin |
| | | | | | | tint; powder primarily | |
| | | | | | | in collection flask, no | |
| | | | | | | build up in cyclone. | |
| 70:30 | 1:1 | 25 | 20:5 | 6.30 | g | White powder, slightly | 30% pectin |
| | | | | | | beige, dry and | |
| | | | | | | granular; normal | |
| | | | | | | distribution on | |
| | | | | | | primary cyclone, most | |
| | | | | | | found in collection | |
| | | | | | | flask. | |
| 70:30 | 1:1 | 25 | 20:5 | 8.72 | g | White slightly beige | X2 water content |
| 70:30 | 1:1 | 25 | 20:5 | 9.30 | g | powder; normal | X2 water content |
| | | | | | | distribution, most | |
| | | | | | | powder in collection | |
| | | | | | | flask | |
| 70:30 | 1:1 | 25 | 20:5 | 10.91 | g | White, slightly beige | X4 water content |
| | | | | | | powder; collection | |
| | | | | | | cyclone and inlets | |
| | | | | | | totally saturated, | |
| | | | | | | primary cyclone | |
| | | | | | | coated. | |
| 70:30 | 1:1 | 25 | 20:5 | 6.15 | g | White powder, dry | X2 water content; |
| | | | | | | and granular; normal | Spray Dryer |
| | | | | | | distribution, most in | Temp: reduced |
| | | | | | | collection flask. | (inlet = 130° C., |
| | | | | | | | outlet = 78° C.) |
| 70:30 | 1:1 | 25 | 20:5 | 6.47 | | White powder, dry | X2 water content; |
| | | | | | | and granular; normal | Increased QFlow |
| | | | | | | distribution, most in | and speed |
| | | | | | | collection flask. | (inlet = 170° C., |
| 70:30 | 1:1 | 25 | 20:5 | 6.55 | | White powder, dry | outlet = 98-103° C.) |
| | | | | | | and granular; normal | |
| | | | | | | distribution, most in | |
| | | | | | | collection flask. | |
| 70:30 | 1:1 | 26 | 20.8:5.2 | 5.84 | | White powder, dry | Increased QFlow |
| | | | | | | and granular; normal | and speed & |
| | | | | | | distribution, most in | heated emulsion |
| | | | | | | collection flask. | feeding |
| 70:30 | 1:1 | 9.78 g + | 7.82:1.96 | 10.10 | | White powder, | Add bulking |
| | | 21.38 g | | | | extremely dry; normal | agent |
| | | myo- | | | | distribution, most in | |
| | | inositol | | | | collection flask. | |
| 70:30 | 1:1 | 25 | 20:5 | 8.25 | | White, slightly | X2 water content; |
| | | | | | | clumpy; normal | Overnight (RT) |
| | | | | | | distribution, most in | wall material |
| | | | | | | collection flask | dissolution |
| 70:30 | 1:1 | 25 | 20:5 | 9.0 | g | White, slightly beige | X2 water content; |
| | | | | | | powder; normal | Emulsion |
| | | | | | | distribution, most in | incubated at RT |
| | | | | | | collection flask | for 44 hours then |
| | | | | | | | mixed 30 mins |
| 70:30 | 1:1 | 25 | 20:5 | 8.35 | g | White, relatively dry | X2 water content; |
| | | | | | | slightly clumpy | 60° C. materials |
| | | | | | | powder, most in | dissolution |
| | | | | | | collection flask | |
| 70:30 | 1:1 | 50 | 40:10 | 5.61 | g | White, slightly beige, | X2 water content; |
| | | | | | | very dry; primary and | QFlow 45, outlet |
| | | | | | | collection cyclones | temp 45-50° C. |
| | | | | | | and inlets totally | (13 minute run) |
| | | | | | | saturated, about 50% | |
| | | | | | | in collection cyclone, | |
| | | | | | | yellow build up inlet | |
| | | | | | | and collection | |
| | | | | | | cyclone, water | |
| | | | | | | condensation at top | |
| | | | | | | of primary cyclone | |
| 70:30 | 1:1 | 50 | 40:10 | 7.57 | g | White, slightly beige | X2 water content; |
| | | | | | | powder, very dry; | QFlow increase |
| | | | | | | primary and collection | to 45, outlet temp |
| | | | | | | cyclones and inlets | 45-50° C. (13 |
| | | | | | | totally saturated, | minute run) |
| | | | | | | about 50% in | & incubate |
| | | | | | | collection cyclone, | emulsion |
| | | | | | | yellow build up inlet | overnight |
| | | | | | | and collection | |
| | | | | | | cyclone, water | |
| | | | | | | condensation at top | |
| | | | | | | of primary cyclone; | |
| | | | | | | yellow cake in | |
| | | | | | | collection cyclone | |
| | | | | | | required breaking up. | |
| 70:30 | 1:1 | 25 | 20:5 | 5.03 | g | White powder, dry but | X2 water content; |
| | | | | | | not granular or | Spray dryer |
| | | | | | | hardened; primary | settings (50% |
| | | | | | | cyclone and inlet | pump rate (about |
| | | | | | | coated, some on | 15 mL/min), |
| | | | | | | collection cyclone, | inlet = 200° C.; |
| | | | | | | yellow cake like build | outlet = 77-70° C., |
| | | | | | | up inlet and collection | 12.5 min run) |
| | | | | | | cyclone, about half in | |
| | | | | | | collection flask. | |
| 70:30 | 1:1 | 25 | 20:5 | 8.79 | g | White, slightly beige | X2 water content; |
| | (CBDi:MCTo) | | | | | powder, dry not | 20% CBDi:MHCo |
| | | | | | | granular; normal | |
| | | | | | | distribution, most in | |
| | | | | | | collection flask. | |
| 70:30 | 1:1 | 26.7 | 20:6.7 | 7.88 | g | White, slightly beige | X2 water content; |
| | (CBDi:MCTo) | | | | | powder, dry not | Active 25% |
| | | | | | | granular; normal | CBDi:MCTo |
| | | | | | | distribution, most in | |
| | | | | | | collection flask. | |
| 70:30 | 1:1 | 28.57 | 20:8.57 | 9.13 | g | White, slightly beige | X2 water content; |
| | (CBDi:MCTo) | | | | | powder, dry not | Active 30% |
| | | | | | | granular; normal | CBDi:MCTo |
| | | | | | | distribution, most in | |
| | | | | | | collection flask. | |
| 70:30 | Pure R-(+)- | 25 | 20:5 | 12.17 | g | White, clumpy | X2 water content; |
| | Limonene | | | | | powder smelling of | Active 20% pure |
| | | | | | | lemons; normal | limonene only |
| | | | | | | distribution, inlets and | & spray dyer |
| | | | | | | collection cyclone | settings (pump |
| | | | | | | clear until H2O purge, | rate at 10% |
| | | | | | | then most powder in | (about |
| | | | | | | collection flask. | 3 mL/min), |
| | | | | | | | inlet = 130° C., |
| | | | | | | | outlet 77-78° C.) |
| 70:30 | Pure R-(+)- | 21.1 | 20:1.1 | 12.20 | g | White, dry, granular | X2 water content; |
| | Limolene | | | | | powder, smelling | Active 5% pure |
| | | | | | | slightly of lemons; | limonene only |
| | | | | | | normal distribution, | |
| | | | | | | inlets and collection | |
| | | | | | | cyclone clear until | |
| | | | | | | H2O purge, then | |
| | | | | | | most powder in | |
| | | | | | | collection flask. | |
| 85:15 | 7:3 | 40.0 | 20:20 | 5.01 | g | White clumpy | Elevated |
| | | | | | | powder, slight yellow | THCd:MCTo |
| | | | | | | tint; powder slightly | ratio |
| | | | | | | damp; normal | |
| | | | | | | distribution of powder | |
| | | | | | | spray drying | |

### Example 11

In this example, encapsulation capacity and stability of spray dried powders comprising different types of pectin in the wall material were compared. A first powder was prepared with pectin sourced from citrus peel and a second powder was prepared with pectin sourced from sugar beet. The powders were prepared according to the methods described in Example 4. The wall materials comprised a 90%:10% w/w ratio of inulin:pectin (9:1 ratio). The core materials comprised a 1:1 mixture of MCT oil and CBD isolate. The ratio of wall:core material was 80%:20% w/w.

TABLE 10

| | Encapsulation |
|---|---|
| Pectin type | efficiency (EE %) |
| Citrus peel | 87.7 |
| Sugar beet | 95.0 |

The sugar beet pectin powder formulation showed a 7% increase in encapsulation efficiency compared to the citrus peel pectin powder formulation (see Table 10). Formulations comprising sugar beet pectin were also found to have better and faster solubility compared to formulations comprising citrus peel pectin. For example, in a side-by-side visual experiment, 200 mg of the spray dried powders were mixed with 250 mL of room temperature tap water. The powder comprising the sugar beet pectin dissolved within 20 seconds of mixing, whereas the spray dried powder comprising citrus pectin had not fully dissolved after 20 seconds.

### Example 12

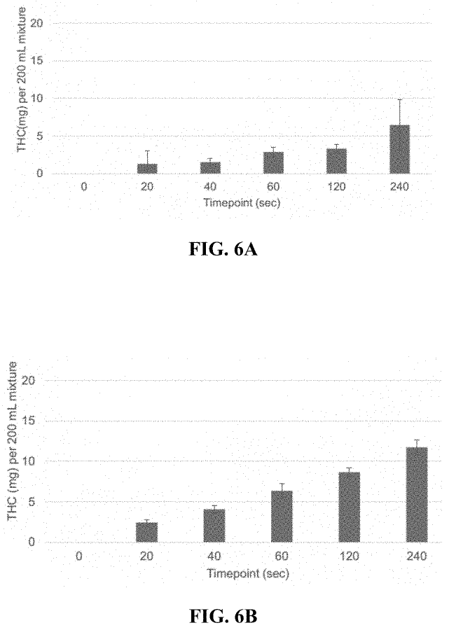

In this example, powder formulations prepared according to the present disclosure were added to a standard tea bag comprising black tea leaves. The powder formulations were prepared according to the methods described in Example 4 and comprised 95%:5% w/w inulin:pectin in the wall materials. Boiling water was added to the tea bags and the beverage was left to steep (brew). The brewed tea was observed visually and the overall THC concentration was assessed over time. Samples were taken at 0, 20, 40, 60, 120 and 240 seconds after addition of boiling water. 2 duplicates were performed. The THC concentration in the steeped tea relative to time are shown in FIG. 6B (compared to a control of the powder only in FIG. 6A).

Overall, the powder-dosed tea bags were effective in producing a beverage with around 10 mg THC after steeping for around 3 minutes (see FIG. 6B).

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

### REFERENCES

[1] Abang Zaidel, D. N., Enzyme catalyzed oxidative cross-linking of feruloylated pectic polysaccharides from sugar beet: Kinetics and rheology. Kgs. Lyngby: Technical University of Denmark, 2012, 1-25.
[2] Funami, T., Zhang, G., Hiroe, M., et al., Effects of the proteinaceous moiety on the emulsifying properties of sugar beet pectin. Food Hydrocolloids, 2007, 21, 1319-1329.
[3] Ngou'mazong, E. D., Christiaens, S., Shpigelman, A., Loey, A. V., Hendrickx, M., The Emulsifying and Emulsion-Stabilizing Properties of Pectin: A Review, Comp. Rev. Food Sci. & Food Saf. 2015, 14, 705-718.
[4] Zhan, D., Janssen, P., & Mort, A. J. Scarcity or complete lack of single rhamnose residues interspersed within the homogalacturonan regions of citrus pectin. Carbohydrate Research, 1998, 308, 373-380.
[5] Voragen, A. G. J., Coenen, G.-J., Verhoef, R. P., & Schols, H. A. Pectin, a versatile polysaccharide present in plant cell walls. Structural Chemistry, 2009, 20, 263-275.
[6] Georgiev, Y., Ogynyanov, M., Yanakieva, I., et al., Isolation, characterization and modification of citrus pectins. J. BioSci. Biotech. 2012, 1(3), 223-233.
[7] Siew, C. K., Williams, P. A., Role of Protein and Ferulic Acid in the Emulsification Properties of Sugar Beet Pectin. J. Agric. Food Chem. 2008, 56, 4164-4171.

1. A composition comprising a cannabinoid or a cannabis-derived compound, inulin and pectin.

2. The composition of claim 1, wherein the inulin and pectin are present in the composition in a ratio of inulin:pectin of between about 99%:1% w/w to about 60%:40% w/w.

3-5. (canceled)

6. The composition of claim 1, wherein the pectin is a citrus pectin.

7. The composition of claim 1, wherein the pectin is a sugar beet pectin.

8. The composition of claim 1, wherein the cannabinoid is Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), Tetrahydrocannabinolic acid C4 (THCA-C4), Tetrahydrocannbinol C4 (THC C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor-9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9-Dihydroxy-Δ6a(10a)-tetrahydrocannabinol (8,9-Di-OH-CBT-C5), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide, or any combination thereof.

9. The composition of claim 1, wherein the cannabinoid is cannabidiol (CBD), tetrahydrocannabinol (THC), or a combination thereof.

10. The composition of claim 1, wherein the cannabis-derived compound is a terpene.

11. (canceled)

12. The composition of claim 1, wherein the composition is a liquid formulation and further comprises a solvent.

13. The composition of claim 12, wherein the solvent is water and the liquid formulation is an emulsion.

14. The composition of claim 1, wherein the composition is a powder formulation.

15-16. (canceled)

17. The composition of claim 1, comprising core-shell structures comprising a hydrophobic inner core comprising the cannabinoid or cannabis-derived compound and a hydrophilic outer shell comprising the inulin and pectin.

18. The composition of claim 17, comprising a ratio of hydrophilic outer shell:hydrophobic inner core of between about 90%:10% w/w to about 50%:50% w/w.

19-20. (canceled)

21. The composition of claim 17, wherein the hydrophobic inner core further comprises a carrier solvent.

22. (canceled)

23. The composition of claim 21, wherein the carrier solvent comprises coconut oil or medium-chain triglyceride (MCT) oil.

24. The composition of claim 21, comprising a weight ratio of cannabinoid:carrier solvent of between about 3:1 to about 1:3.

25-26. (canceled)

27. The composition according to claim 1, comprising core-shell structures comprising a hydrophobic inner core comprising the cannabinoid and a hydrophilic outer shell comprising the inulin and pectin, wherein:
the inulin and pectin are present in a ratio of about 70%:30% w/w inulin:pectin;
the core-shell structures comprise a ratio of about 80%:20% w/w hydrophilic outer shell:hydrophobic inner core; and
the hydrophobic inner core further comprises a carrier solvent at a ratio of about 1:1 w/w cannabinoid:carrier solvent.

28. (canceled)

29. A method of preparing the composition of claim 1, the method comprising: combining a cannabinoid or a cannabis-derived compound with inulin and pectin, and homogenizing the mixture with a solvent to form an emulsion.

30. The method of claim 29, which comprises:
combining the inulin and pectin in the solvent to form an inulin/pectin mixture;
combining the cannabinoid or the cannabis-derived compound with the inulin/pectin mixture; and
homogenizing the mixture to form an emulsion.

31. The method according to claim 30, further comprising mixing the cannabinoid or the cannabis-derived compound in a carrier solvent prior to combining the cannabinoid or the cannabis-derived compound with the inulin/pectin mixture.

32. The method according to claim 31, wherein the carrier solvent comprises coconut oil or medium-chain triglyceride (MCT) oil.

33-35. (canceled)

36. The method according to claim 29, further comprising drying the emulsion to form a powder.

37. (canceled)

38. The method according to claim 36, wherein the powder comprises core-shell structures comprising a hydrophobic inner core comprising the cannabinoid or cannabis-derived compound and a hydrophilic outer shell comprising the inulin and pectin.

39. The method according to claim 38, wherein at least 50% by weight of total the cannabinoid or cannabis-derived compound that was combined with the inulin and pectin is encapsulated in the hydrophobic inner core.

40. (canceled)

41. A composition prepared according to the method of claim 29.

42. A foodstuff comprising the composition of claim 1.

43. A beverage comprising the composition of claim 1.
